(19) United States
(12) Patent Application Publication
(10) Pub. No.: US 2022/0370711 A1
(43) Pub. Date: Nov. 24, 2022

(54) TRANSFUSION PUMP WITH AN INSERTION DEVICE

(71) Applicant: E3D A.C.A.L., Merom Hagalil (IL)

(72) Inventors: Ziv NAFTALOVITZ, West Galilee (IL); Tsachi SHAKED, Merom Hagalil (IL); Ozi BEN ARI, Upper Galilee (IL); Michael SEGEV, Lower Galilee (IL); Ilan SHOPEN, Zefat (IL)

(73) Assignee: E3D A.C.A.L., Merom Hagalil (IL)

(21) Appl. No.: 17/783,944

(22) PCT Filed: Dec. 17, 2020
(86) PCT No.: PCT/IL2020/051299

§ 371 Date: Jun. 9, 2022

Related U.S. Application Data
(60) Provisional application No. 62/949,449, filed on Dec. 18, 2019.

(51) Int. Cl. A61M 5/145; A61M 5/142

(52) U.S. Cl. CPC A61M 5/14526; A61M 5/14248; A61M 2005/14252; A61M 2005/14204

(57) ABSTRACT

A pump assembly for pumping a medication through a cannula, said pump assembly comprising a housing adapted to be attached to a skin of a patient and containing a reservoir assembly having a variable volume liquid chamber configured to contain the medication upon filling of the reservoir assembly, a variable volume gas chamber configured to contain air before filling of the reservoir assembly and a pressure chamber configured to contain a pressure generating element, the liquid chamber, gas chamber and pressure chamber are fluidly sealed with respect to each other; the liquid chamber is selectably fluidly couplable with the cannula and the gas chamber is fluidly couplable with the atmosphere.

### REFERENCE TO RELATED APPLICATIONS

Reference is hereby made to U.S. Provisional Patent Application 62/949,449, filed Dec. 18, 2019 and entitled “TRANSFUSION PUMP WITH AN INSERTION DEVICE”, the disclosure of which is incorporated by reference in its entirety and priority of which is hereby claimed pursuant to 37 CFR 1.78(a) (4) and (5)(i).

Reference is also hereby made to PCT Patent Application PCT/IL18/50053, filed Jan. 15, 2018 and entitled “TRANSFUSION PUMP”, the disclosure of which is incorporated by reference in its entirety.

### FIELD OF THE INVENTION

The present invention relates to medicament transfusion and injection devices generally.

### BACKGROUND OF THE INVENTION

Various types of medicament transfusion and injection devices are known in the art.

### SUMMARY OF THE INVENTION

The present invention seeks to provide an improved transfusion pump and a method of use thereof.

There is thus provided in accordance with an embodiment of the present invention a pump assembly for pumping a medication through a cannula, the pump assembly comprising a housing adapted to be attached to a skin of a patient and containing a reservoir assembly having a variable volume liquid chamber configured to contain the medication upon filling of the reservoir assembly, a variable volume gas chamber configured to contain air before filling of the reservoir assembly and a pressure chamber configured to contain a pressure generating element, the liquid chamber, gas chamber and pressure chamber are fluidly sealed with respect to each other; the liquid chamber is selectably fluidly couplable with the cannula and the gas chamber is fluidly couplable with the atmosphere.

Preferably, a first piston assembly and a second piston assembly being slidably sealingly disposed within the reservoir assembly and wherein the liquid chamber is defined between the second piston assembly and a closed forward end of the reservoir assembly and the gas chamber is defined between the first piston assembly and the second piston assembly. Further preferably, the pump assembly also comprising a circuit and switch assembly operative for electronically controlling the pressure generating element. Still further preferably, the second piston assembly has an actuating element that extends forwardly through the closed forward end and being operative for electrically coupling the pressure generating element to the electronic assembly upon driving of the at least one of the first piston assembly and the second piston assembly in a medication filling displacement direction. Yet further preferably, at least one of the first piston assembly and the second piston assembly is configured to be driven in a medication pumping displacement direction by fluid pressure generated by the pressure generating element upon receipt of a suitable signal from the circuit and switch assembly.

In accordance with an embodiment of the present invention, the second piston assembly abuts the closed forward end before filling of the liquid chamber with the medication. Preferably, the first and second piston assemblies both move in a same axial direction during pumping of the medication through the cannula. Further preferably, the pump assembly is adapted for pumping the medication through the cannula at a given time, and wherein the given time is measured from supplying the medication into the reservoir assembly and thereby displacing the second piston assembly rearwardly within the reservoir assembly.

Still further preferably, the pump assembly also comprising an inserter removably coupled with the pump assembly and having a penetrating element, which is pivotably coupled to the inserter and is configured to extend within the cannula for insertion of the cannula into an injection site; the penetrating element is pivotable between a retracted position within the inserter and an extended position within the cannula, the penetrating element being biased to the retracted position following removal thereof from the cannula.

Yet further preferably, the actuating element fluidly couples the gas chamber with the atmosphere.

In accordance with an embodiment of the present invention, a first fluid path is operative for passage of the medicament between the liquid chamber and the cannula and a second fluid path is operative for passage or air between the gas chamber and the atmosphere, and wherein the first fluid path and the second fluid path are fluidly sealed with respect to each other. Preferably, the first fluid path, the second fluid path, the liquid chamber and the gas chamber are accessible for sterilization by a sterilizing agent. Further preferably, the circuit and switch assembly comprises a sensor, which is configured for providing an indication of an empty liquid chamber upon engagement between the sensor and the actuating element.

In accordance with an embodiment of the present invention, the pump assembly comprising a housing configured for enclosing a reservoir assembly therewithin, the reservoir assembly having a closed forward end, disposed adjacent the cannula; an electronic assembly, operatively engageable with the reservoir assembly; the reservoir assembly comprises a first piston assembly, a second piston assembly and a pressure generating element enclosed therewithin, the at least one of the first piston assembly and the second piston assembly has an actuating element that extends forwardly through the closed forward end and being operative for electrically coupling the pressure generating element to the electronic assembly upon driving of the at least one of the first piston assembly and the second piston assembly in a medication filling displacement direction.

Preferably, the housing is adapted to be attached to a skin of a patient and the reservoir assembly having a variable volume liquid chamber configured to contain the medication upon filling of the reservoir assembly, a variable volume gas chamber configured to contain air before filling of the reservoir assembly and a pressure chamber configured to contain the pressure generating element, the liquid chamber, gas chamber and pressure chamber are fluidly sealed with respect to each other; the liquid chamber is selectably fluidly couplable with the cannula and the gas chamber is fluidly couplable with the atmosphere. Further preferably, the first piston assembly and the second piston assembly being slidably sealingly disposed within the reservoir assembly and wherein the liquid chamber is defined between the second piston assembly and the closed forward end of the reservoir assembly and the gas chamber is defined between the first piston assembly and the second piston assembly.

In accordance with an embodiment of the present invention, the electronic assembly being operative for electronically controlling the pressure generating element. Preferably, at least one of the first piston assembly and the second piston assembly is configured to be driven in a medication pumping displacement direction by fluid pressure generated by the pressure generating element upon receipt of a suitable signal from the electronic assembly. Further preferably, the second piston assembly abuts the closed forward end before filling of the liquid chamber with the medication.

Still further preferably, the first and second piston assemblies both move in a same axial direction during pumping of the medication through the cannula. Yet further preferably, the pump assembly is adapted for pumping the medication through the cannula at a given time, and wherein the given time is measured from supplying the medication into the reservoir assembly and thereby displacing the second piston assembly rearwardly within the reservoir assembly.

In accordance with an embodiment of the present invention, the pump assembly also comprising an inserter removably coupled with the pump assembly and having a penetrating element, which is pivotably coupled to the inserter and is configured to extend within the cannula for insertion of the cannula into an injection site; the penetrating element is pivotable between a retracted position within the inserter and an extended position within the cannula, the penetrating element being biased to the retracted position following removal thereof from the cannula.

Preferably, the actuating element fluidly couples the gas chamber with the atmosphere. Further preferably, a first fluid path is operative for passage of the medicament between the liquid chamber and the cannula and a second fluid path is operative for passage or air between the gas chamber and the atmosphere, and wherein the first fluid path and the second fluid path are fluidly sealed with respect to each other. Still further preferably, the first fluid path, the second fluid path, the liquid chamber and the gas chamber are accessible for sterilization by a sterilizing agent. Yet further preferably, the electronic assembly comprises a sensor, which is configured for providing an indication of an empty liquid chamber upon engagement between the sensor and the actuating element.

In accordance with an embodiment of the present invention, a pump assembly useful for pumping a medication through a cannula, the pump assembly comprising a housing configured for enclosing a reservoir assembly therewithin, the reservoir assembly having a closed forward end, disposed adjacent the cannula; an electronic assembly, operatively engageable with the reservoir assembly; the reservoir assembly comprises a first piston assembly, a second piston assembly and a pressure generating element enclosed therewithin, a liquid chamber is configured to be formed between the second piston assembly and the closed forward end; a gas chamber is configured to be formed between the second piston assembly and the first piston assembly and a pressure chamber is configured to be formed between the pressure generating element and the first piston assembly, wherein at least one of the first piston assembly and the second piston assembly is configured to be driven in a medication pumping displacement direction by fluid pressure generated by the pressure generating element upon receipt of a suitable signal from the electronic assembly.

Preferably, the at least one of the first piston assembly and the second piston assembly has an actuating element that extends forwardly through the closed forward end and being operative for electrically coupling the pressure generating element to the electronic assembly upon driving of the at least one of the first piston assembly and the second piston assembly in a medication filling displacement direction.

Further preferably, the housing is adapted to be attached to a skin of a patient and the liquid chamber has a variable volume configured to contain the medication upon filling of the reservoir assembly, the gas chamber has a variable volume configured to contain air before filling of the reservoir assembly and the pressure chamber has a variable volume configured to contain the pressure generating element, the liquid chamber, gas chamber and pressure chamber are fluidly sealed with respect to each other; the liquid chamber is selectably fluidly couplable with the cannula and the gas chamber is fluidly couplable with the atmosphere.

Still further preferably, the first piston assembly and the second piston assembly being slidably sealingly disposed within the reservoir assembly. Yet further preferably, the electronic assembly being operative for electronically controlling the pressure generating element.

In accordance with an embodiment of the present invention, the second piston assembly abuts the closed forward end before filling of the liquid chamber with the medication. Preferably, the first and second piston assemblies both move in a same axial direction during pumping of the medication through the cannula. Further preferably, the pump assembly is adapted for pumping the medication through the cannula at a given time, and wherein the given time is measured from supplying the medication into the reservoir assembly and thereby displacing the second piston assembly rearwardly within the reservoir assembly.

Still further preferably, the pump assembly also comprising an inserter removably coupled with the pump assembly and having a penetrating element, which is pivotably coupled to the inserter and is configured to extend within the cannula for insertion of the cannula into an injection site; the penetrating element is pivotable between a retracted position within the inserter and an extended position within the cannula, the penetrating element being biased to the retracted position following removal thereof from the cannula. Yet further preferably, the actuating element fluidly couples the gas chamber with the atmosphere.

In accordance with an embodiment of the present invention, a first fluid path is operative for passage of the medicament between the liquid chamber and the cannula and a second fluid path is operative for passage or air between the gas chamber and the atmosphere, and wherein the first fluid path and the second fluid path are fluidly sealed with respect to each other. Preferably, the first fluid path, the second fluid path, the liquid chamber and the gas chamber are accessible for sterilization by a sterilizing agent. Further preferably, the electronic assembly comprises a sensor, which is configured for providing an indication of an empty liquid chamber upon engagement between the sensor and the actuating element.

In accordance with an embodiment of the present invention, a pump assembly for pumping a medication through a cannula, the pump assembly comprising a timed pumping assembly adapted for pumping the medication through the cannula at a given time; an inserter removably coupled with the timed pumping assembly and having a penetrating element, which is pivotably coupled to the inserter and is configured to extend within the cannula for insertion of the cannula into an injection site; the penetrating element is pivotable between a retracted position within the inserter and an extended position within the cannula, the penetrating element being biased to the retracted position following removal thereof from the cannula.

Preferably, the pump assembly also comprising a housing configured for enclosing a reservoir assembly therewithin, the reservoir assembly having a closed forward end, disposed adjacent the cannula; an electronic assembly, operatively engageable with the reservoir assembly; the reservoir assembly comprises a first piston assembly, a second piston assembly and a pressure generating element enclosed therewithin, a liquid chamber is configured to be formed between the second piston assembly and the closed forward end; a gas chamber is configured to be formed between the second piston assembly and the first piston assembly and a pressure chamber is configured to be formed between the pressure generating element and the first piston assembly, wherein at least one of the first piston assembly and the second piston assembly is configured to be driven in a medication pumping displacement direction by fluid pressure generated by the pressure generating element upon receipt of a suitable signal from the electronic assembly.

Further preferably, the at least one of the first piston assembly and the second piston assembly has an actuating element that extends forwardly through the closed forward end and being operative for electrically coupling the pressure generating element to the electronic assembly upon driving of the at least one of the first piston assembly and the second piston assembly in a medication filling displacement direction. Still further preferably, the housing is adapted to be attached to a skin of a patient and the liquid chamber has a variable volume configured to contain the medication upon filling of the reservoir assembly, the gas chamber has a variable volume configured to contain air before filling of the reservoir assembly and the pressure chamber has a variable volume configured to contain the pressure generating element, the liquid chamber, gas chamber and pressure chamber are fluidly sealed with respect to each other; the liquid chamber is selectably fluidly couplable with the cannula and the gas chamber is fluidly couplable with the atmosphere.

In accordance with an embodiment of the present invention, the first piston assembly and the second piston assembly being slidably sealingly disposed within the reservoir assembly. Preferably, the electronic assembly being operative for electronically controlling the pressure generating element. Further preferably, the second piston assembly abuts the closed forward end before filling of the liquid chamber with the medication.

Still further preferably, the first and second piston assemblies both move in a same axial direction during pumping of the medication through the cannula. Yet further preferably, the pump assembly is adapted for pumping the medication through the cannula at a given time, and wherein the given time is measured from supplying the medication into the reservoir assembly and thereby displacing the second piston assembly rearwardly within the reservoir assembly.

In accordance with an embodiment of the present invention, the actuating element fluidly couples the gas chamber with the atmosphere. Preferably, a first fluid path is operative for passage of the medicament between the liquid chamber and the cannula and a second fluid path is operative for passage or air between the gas chamber and the atmosphere, and wherein the first fluid path and the second fluid path are fluidly sealed with respect to each other. Further preferably, the first fluid path, the second fluid path, the liquid chamber and the gas chamber are accessible for sterilization by a sterilizing agent. Still further preferably, the electronic assembly comprises a sensor, which is configured for providing an indication of an empty liquid chamber upon engagement between the sensor and the actuating element.

In accordance with an embodiment of the present invention, a pump assembly for pumping a medication through a cannula, the pump assembly comprising a housing adapted to be attached to a skin of a patient and containing a reservoir assembly having a variable volume liquid chamber configured to contain the medication upon filling of said reservoir assembly, a variable volume gas chamber configured to contain air before filling of the reservoir assembly and a pressure chamber configured to contain a pressure generating element, the liquid chamber, gas chamber and pressure chamber are fluidly sealed with respect to each other; the volume of the liquid chamber is mutually variable with the volume of the gas chamber during filling of the liquid chamber with the medication, such that the volume increase of the liquid chamber corresponds to volume decrease of the gas chamber; the volume of the liquid chamber is mutually variable with the volume of the pressure chamber during pumping of the medication out of the liquid chamber through the cannula, such that the volume increase of the pressure chamber corresponds to volume decrease of the liquid chamber.

Preferably, a first piston assembly is provided between the pressure chamber and the gas chamber; a second piston assembly is provided between the gas chamber and the liquid chamber and the first and second piston assemblies are configured to be slidably sealingly displaceable within the reservoir assembly. Further preferably, the pump assembly also comprising an electronic assembly, operatively engageable with the reservoir assembly, the reservoir assembly having a closed forward end, disposed adjacent the cannula; the reservoir assembly comprises the first piston assembly, the second piston assembly and a pressure generating element enclosed therewithin, said liquid chamber is configured to be formed between the second piston assembly and the closed forward end; the gas chamber is configured to be formed between the second piston assembly and the first piston assembly and the pressure chamber is configured to be formed between the pressure generating element and the first piston assembly, wherein at least one of the first piston assembly and the second piston assembly is configured to be driven in a medication pumping displacement direction by fluid pressure generated by the pressure generating element upon receipt of a suitable signal from the electronic assembly.

Still further preferably, the at least one of the first piston assembly and the second piston assembly has an actuating element that extends forwardly through the closed forward end and being operative for electrically coupling the pressure generating element to the electronic assembly upon driving of the at least one of the first piston assembly and the second piston assembly in a medication filling displacement direction. Yet further preferably, the liquid chamber is selectably fluidly couplable with the cannula and the gas chamber is fluidly couplable with the atmosphere.

In accordance with an embodiment of the present invention, the electronic assembly being operative for electronically controlling the pressure generating element. Preferably, the second piston assembly abuts the closed forward end before filling of the liquid chamber with the medication. Further preferably, the first and second piston assemblies both move in a same axial direction during pumping of the medication through the cannula. Still further preferably, the pump assembly is adapted for pumping the medication through the cannula at a given time, and wherein the given time is measured from supplying the medication into the reservoir assembly and thereby displacing the second piston assembly rearwardly within the reservoir assembly. Yet further preferably, the actuating element fluidly couples the gas chamber with the atmosphere.

In accordance with an embodiment of the present invention, a first fluid path is operative for passage of the medicament between the liquid chamber and the cannula and a second fluid path is operative for passage or air between the gas chamber and the atmosphere, and wherein the first fluid path and the second fluid path are fluidly sealed with respect to each other. Preferably, the first fluid path, the second fluid path, the liquid chamber and the gas chamber are accessible for sterilization by a sterilizing agent.

Further preferably, the electronic assembly comprises a sensor, which is configured for providing an indication of an empty liquid chamber upon engagement between the sensor and the actuating element. Still further preferably, the pump assembly also comprising an inserter removably coupled with the pump assembly and having a penetrating element, which is pivotably coupled to the inserter and is configured to extend within the cannula for insertion of the cannula into an injection site; the penetrating element is pivotable between a retracted position within the inserter and an extended position within the cannula, the penetrating element being biased to the retracted position following removal thereof from the cannula.

### BRIEF DESCRIPTION OF THE DRAWINGS

The present invention will be understood and appreciated more fully from the following detailed description, taken in conjunction with the drawings in which:

FIG. 1 is a simplified pictorial illustration of a transfusion pump with a filling port mounted thereto and an inserter associated therewith, constructed and operative in accordance with an embodiment of the present invention;

FIGS. 2A & 2B are respective simplified exploded pictorial and sectional illustrations of the transfusion pump of FIG. 1, section being taken along lines B-B in FIG. 2A;

FIGS. 3A-3C are respective simplified pictorial illustration and two sectional illustrations taken along perpendicular lines B-B and C-C in FIG. 3A of an inserter associated with the transfusion pump of FIG. 1;

FIG. 4 is a simplified pictorial illustration of a penetrating element associated with the inserter of FIGS. 3A-3C;

FIGS. 5A, 5B and 5C are respective simplified pictorial and two sectional illustrations of a top housing portion of the transfusion pump of FIG. 1, sections being taken along perpendicular lines B-B and C-C in FIG. 5A;

FIG. 6 is a simplified pictorial illustration of a reservoir assembly, forming part of the transfusion pump of FIG. 1;

FIGS. 7A-7C are respective simplified exploded illustration and two sectional illustrations of the reservoir assembly of FIG. 6, sections being taken along lines B-B and C-C in FIG. 7A;

FIGS. 8A and 8B are respective simplified pictorial illustration and sectional view of a barrel, forming part of the reservoir assembly of FIGS. 6-7C, section being taken along lines B-B in FIG. 8A;

FIGS. 9A and 9B are respective simplified pictorial illustration and sectional view of a first piston assembly, forming part of the reservoir assembly of FIGS. 6-7C, section being taken along lines B-B in FIG. 9A;

FIGS. 10A and 10B are respective simplified pictorial illustration and sectional view of a second piston assembly, forming part of the reservoir assembly of FIGS. 6-7C, section being taken along lines B-B in FIG. 10A;

FIGS. 11A-11C are respective simplified pictorial illustration and two sectional views of a reservoir lid, forming part of the reservoir assembly of FIGS. 6-7C, sections being taken along lines B-B and C-C in FIG. 11A;

FIGS. 12A and 12B are two simplified sectional views of the reservoir assembly of FIG. 6, sections being taken along lines A-A and B-B in FIG. 6;

FIGS. 13A-13C are respective simplified pictorial illustration, top view and a sectional view of a flow path septum, forming part of the transfusion pump of FIG. 1, section being taken along lines C-C in FIG. 13B;

FIGS. 14A-14C are respective simplified pictorial illustration, top view and a sectional view of a cannula, forming part of the transfusion pump of FIG. 1, section being taken along lines C-C in FIG. 14A;

FIG. 15 is a simplified pictorial illustration of a circuit and switch assembly, forming part of the transfusion pump of FIG. 1;

FIGS. 16A and 16B are respective simplified pictorial illustration and sectional view of a bottom housing portion of the transfusion pump of FIG. 1, section being taken along lines B-B in FIG. 16A;

FIGS. 17A and 17B are respective simplified top view and pictorial sectional view illustrations of a filling port associated with the transfusion pump of FIG. 1, section being taken along lines B-B in FIG. 17A;

FIGS. 18A-18D are respective simplified pictorial illustration and three sectional views of the transfusion pump of FIG. 1 shown in a first operative orientation, sections being taken along lines B-B, C-C and D-D in FIG. 18A;

FIGS. 19A and 19B are respective simplified pictorial illustration and sectional view of the transfusion pump of FIG. 1 shown in a second operative orientation, section being taken along lines B-B in FIG. 19A;

FIGS. 20A-20D are respective simplified pictorial illustration and three sectional views of the transfusion pump of FIG. 1 shown in a third operative orientation, sections being taken along lines B-B, C-C and D-D in FIG. 20A;

FIGS. 21A and 21B are respective simplified pictorial illustration and sectional view of the transfusion pump of FIG. 1 shown in a fourth operative orientation, section being taken along lines B-B in FIG. 21A;

FIGS. 22A and 22B are respective simplified pictorial illustration and sectional view of the transfusion pump of FIG. 1 shown in a fifth operative orientation, section being taken along lines B-B in FIG. 22A;

FIGS. 23A and 23B are respective simplified pictorial illustration and sectional view of the transfusion pump of FIG. 1 shown in a sixth operative orientation, section being taken along lines B-B in FIG. 23A;

FIGS. 24A-24C are respective simplified pictorial illustration and two sectional views of the transfusion pump of FIG. 1 shown in a seventh operative orientation, sections being taken along lines B-B and C-C in FIG. 24A.

### DETAILED DESCRIPTION OF EMBODIMENTS OF INVENTION

Described below in accordance with an embodiment of the present invention is a patch transfusion pump assembly, which is adapted to be secured to a patient body surface and is useful for pumping a medicament through a medicament injection pathway into a patient body. The pump assembly generally includes: a timed pumping assembly adapted for pumping the medicament through a cannula at a given time and an inserter operatively coupled with the timed pumping assembly and having a penetration element, which is configured to facilitate cannula penetration into the patient's body. Once the inserter is decoupled from the timed pumping assembly, the medicament can be pumped by the time pumping assembly through the medicament injection pathway and through the cannula.

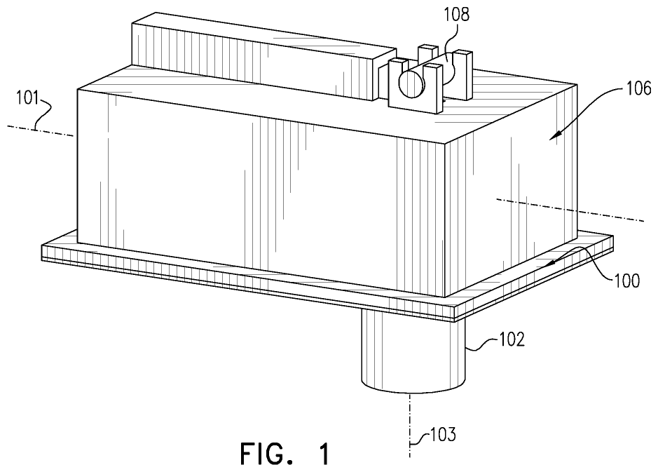

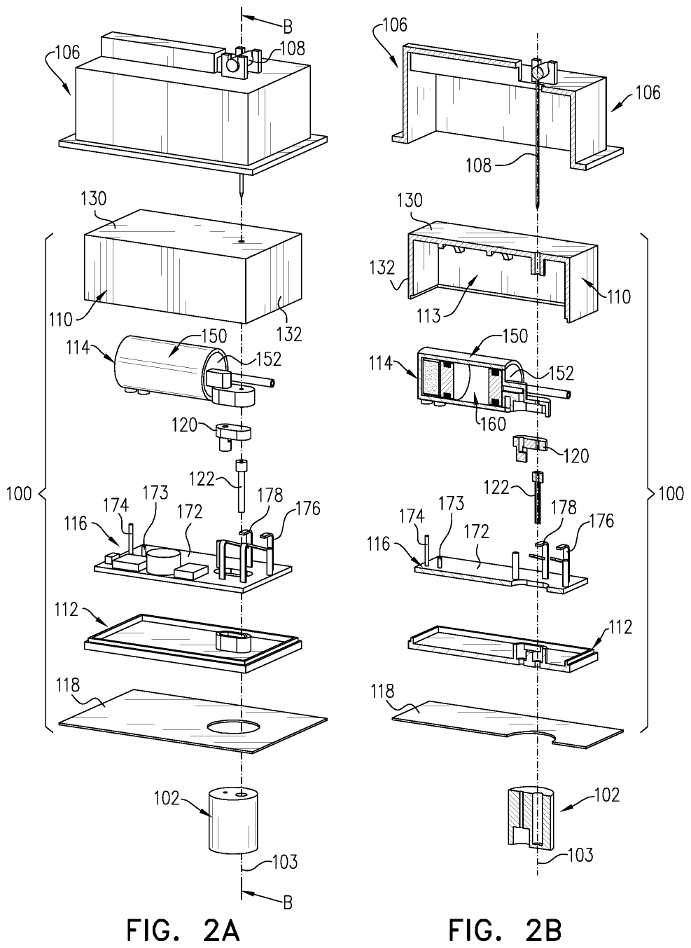

Reference is now made to FIG. 1, which is a simplified pictorial illustration of a transfusion pump with a filling port mounted thereto and an inserter associated therewith, constructed and operative in accordance with a preferred embodiment of the present invention. Reference is additionally made to FIGS. 2A & 2B, which are respective simplified exploded pictorial and sectional illustrations of the transfusion pump of FIG. 1, section being taken along lines B-B in FIG. 2A.

As seen in FIG. 1, a transfusion pump 100 is generally arranged along a longitudinal axis 101 and a filling port 102 is mounted onto the transfusion pump 100 and is arranged along an axis 103, which extends generally perpendicularly with respect to axis 101. It is noted that in an “out of the box” operative orientation, the filling port 102 is removably mounted onto the transfusion pump 100 and an inserter 106 having a penetrating element 108 coupled thereto is removably mounted onto the transfusion pump 100 as well. The transfusion pump 100 is generally also termed as pump assembly.

As seen in FIGS. 2A & 2B, transfusion pump 100 preferably includes a top housing portion 110, and a bottom housing portion 112 configured to be fixedly mounted to the top housing portion 110 and define an interior volume 113 therebetween, which encloses a reservoir assembly 114 and a circuit and switch assembly 116. An adhesive layer 118 is adapted to at least partially cover the underside of the bottom housing portion 112. The filling port 102 is partially inserted through the bottom housing portion 112 and extends into interior volume 113 formed between the top housing portion 110 and the bottom housing portion 112 of the transfusion pump 100. A flow path septum 120 is adapted to reside between the reservoir assembly 114 and the bottom housing portion 112 and a cannula 122 which serves as at least a portion of the medicament fluid pathway into the patient's body is preferably seated and supported at least by a portion of the bottom housing portion 112.

It is particularly seen in FIGS. 2A & 2B that the top housing portion 110 generally has a flat wall portion 130 and a circumferential wall 132 extending downwardly generally transversely thereto. The outer circumference of the bottom housing portion 112 is suited to fit snugly within the top housing portion 110.

The reservoir assembly 114 generally resides within the interior volume 113. The reservoir assembly 114 generally includes a barrel 150 having a lid 152 adapted to close the forward end thereof. A pressure generating assembly 160 is at least partially enclosed within the reservoir assembly 114.

It is a particular feature of an embodiment of the present invention that the circuit and switch assembly 116 includes a printed circuit board 172, which has electrical contacts 173 and 174, which are selectively operatively connected with at least a portion of the pressure generating assembly 160. It is noted that the circuit and switch assembly 116 also includes typically two switches 176 and 178, which are operative for selectively coupling the electrical contacts 173 and 174 with the pressure generating assembly 160.

The filling port assembly 102 is partially inserted through the bottom housing portion 112 and includes an opening for insertion of the cannula 122 therethrough and an opening adapted for insertion of a needle of a pre-filled syringe (not shown), as described in detail hereinbelow.

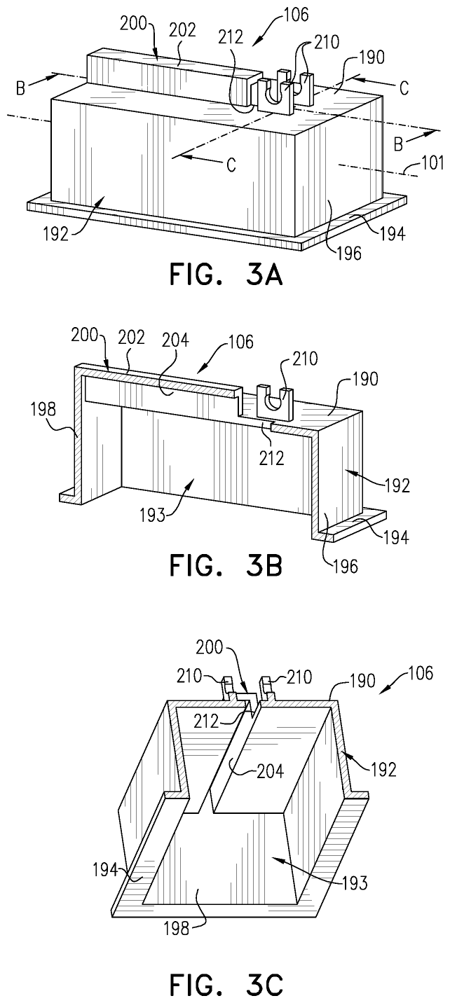

Reference is now made to FIGS. 3A-3C, which are respective simplified pictorial illustration and two sectional illustrations taken along perpendicular lines B-B and C-C in FIG. 3A of the inserter 106 associated with the transfusion pump 100 of FIG. 1.

The inserter 106 is preferably integrally formed from plastic and is generally arranged along longitudinal axis 101.

It is seen in FIGS. 3A-3C that inserter 106 generally has a flat wall portion 190 and a circumferential wall 192 extending downwardly generally transversely thereto and defining an inner volume 193. A generally circumferential flange 194 preferably extends outwardly from the downwardly facing circumferential edge of wall 192. The circumferential wall 192 includes a forward wall portion 196 and a rearward wall portion 198. A generally hollow protrusion 200 is formed on the flat wall portion 190 and is generally located adjacent rearward wall portion 198 and extends forwardly towards forward wall portion 196. The protrusion 200 defines a wall portion 202, which is generally parallel to and spaced from wall portion 190. A groove 204 is formed in wall portion 190 and extends into protrusion 200.

It is also seen in FIGS. 3A-3C that typically two hooks 210 extend upwardly from wall portion 190 and disposed generally between protrusion 200 and forward wall portion 196. An opening 212 is formed in wall portion 190 generally between hooks 210 and protrusion 200.

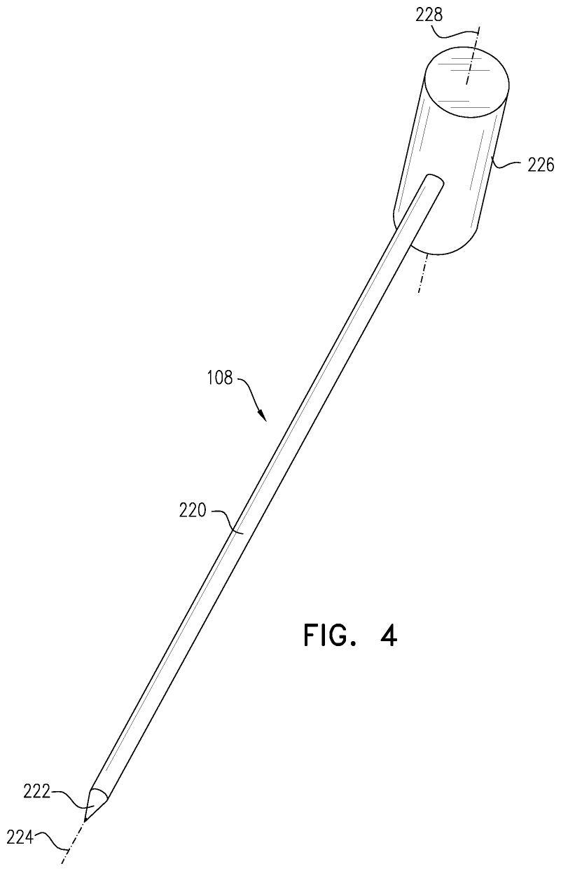

Reference is now made to FIG. 4, which is a simplified pictorial illustration of the penetrating element 108 associated with the inserter 106 of FIGS. 3A-3C.

The penetrating element 108 is preferably integrally formed from metal and includes a longitudinal portion 220 having a sharp end 222 and extending along longitudinal axis 224 and an axle 226 formed at the opposite end of the longitudinal portion 220 and extending along axis 228, which is disposed generally transversely with respect to axis 224.

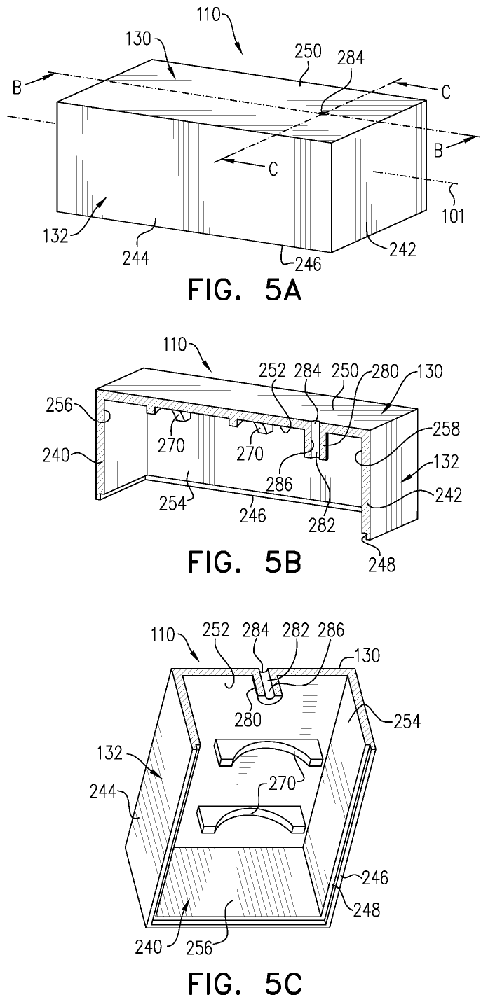

Reference is now made to FIGS. 5A, 5B and 5C, which are respective simplified pictorial and two sectional illustrations of the top housing portion 110 of the transfusion pump 100 of FIG. 1, sections being taken along perpendicular lines B-B and C-C in FIG. 5A.

The top housing portion 110 is preferably integrally formed from plastic and is generally arranged along longitudinal axis 101.

It is seen in FIGS. 5A-5C and mentioned hereinabove that top housing portion 110 generally has flat wall portion 130 and circumferential wall 132 extending downwardly generally transversely thereto. Interior volume 113 is defined by wall portion 130 and circumferential wall 132.

The circumferential wall 132 includes a generally flat rearward wall portion 240, a forward generally flat wall portion 242 and two side walls 244 generally spaced from each other, each connecting the rearward wall portion 240 with the forward wall portion 242. The circumferential wall 132 defines a downwardly facing circumferential edge 246. A circumferential recess is formed on the inner surface of the circumferential wall 132 adjacent the circumferential edge 246 and defines a circumferential downwardly facing shoulder 248, which is slightly upwardly spaced from the circumferential edge 246.

The flat wall portion 130 defines an outer surface 250 and an inner surface 252, the side walls 244 each define an inner surface 254, the rearward wall portion 240 defines an inner surface 256 and the forward wall portion 242 defines an inner surface 258.

It is noted that a window for visual inspection of the contents of the reservoir assembly 114 may be formed in flat wall portion 130. Additionally, a LED opening or alternatively, a transparent portion may be formed on the top housing portion 110, typically on flat wall portion 130.

It is seen in FIGS. 5A-5C that typically two axially spaced generally arcuate protrusions 270 are formed on the flat wall portion 130 and extend generally downwardly from the inner surface 252 thereof. The arcuate protrusions 270 are generally spaced apart along longitudinal axis 101 and are adapted for supporting an upward portion of the reservoir assembly 114.

A protrusion 280 is formed on wall portion 130 and extends generally downwardly from the inner surface 252 thereof into the interior volume 113 of the top housing portion 110. The protrusion 280 is preferably disposed adjacent the forward wall portion 242 and defines a through bore 282, which also extends through wall portion 130 and forms an opening 284 therein. The through bore 282 defines an inner circumferential surface 286.

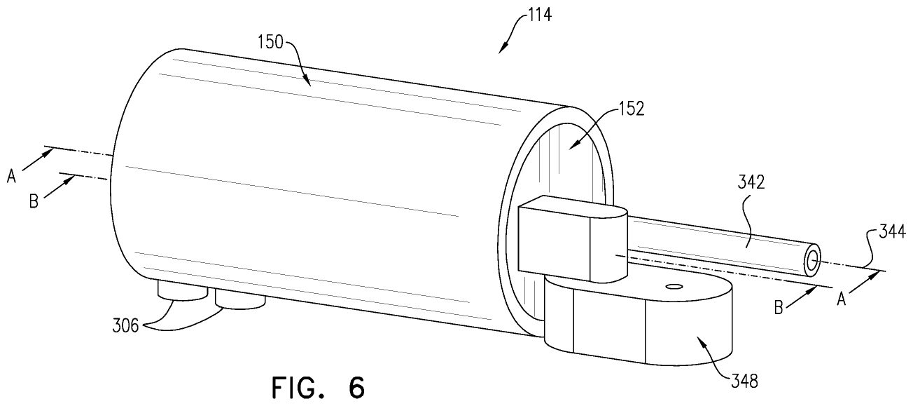

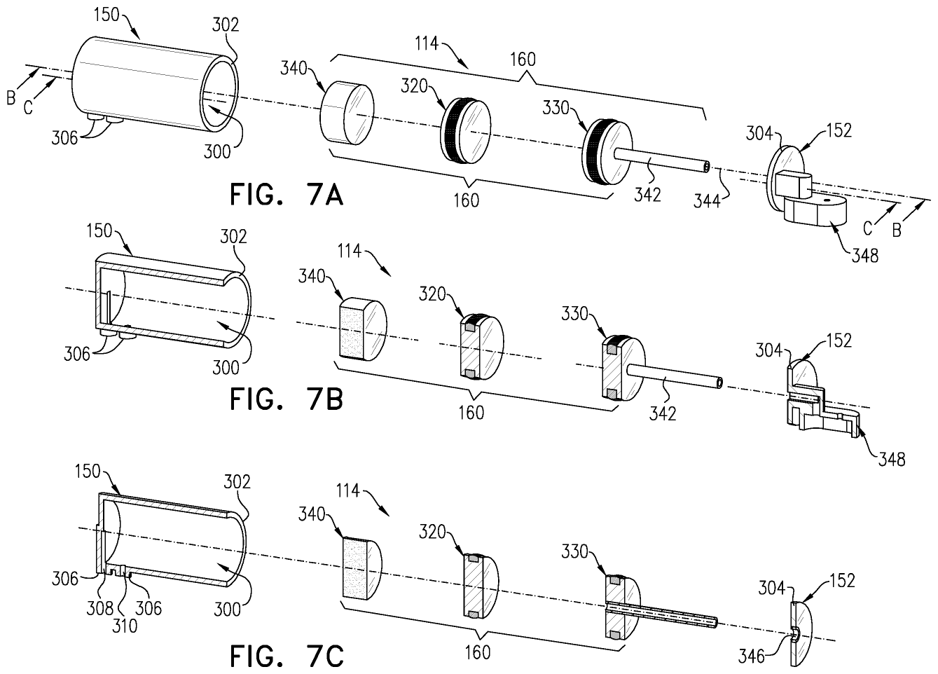

Reference is now made to FIG. 6, which is a simplified pictorial illustration of the reservoir assembly 114, forming part of the transfusion pump 100 of FIG. 1 and to FIGS. 7A-7C, which are respective simplified exploded illustration and two sectional illustrations of the reservoir assembly 114 of FIG. 6, sections being taken along lines B-B and C-C in FIG. 7A.

The reservoir assembly 114 is seen in FIG. 6. As mentioned hereinabove, the reservoir assembly 114 generally includes barrel 150 having the lid 152 adapted to close the forward end thereof. The pressure generating assembly 160 is at least partially enclosed within the reservoir assembly 114.

It is particularly seen in FIGS. 7A-7C that the reservoir assembly 114 includes barrel 150 that defines an interior volume 300 and a forwardly facing circumferential edge 302. The lid 152 has a generally circular flange 304, which is adapted to be fixedly attached to circumferential edge 302, for example by means of pressure-fit engagement or by means of ultrasonic welding. The barrel 150 further includes typically two protrusions 306, each having a respective internal socket 308 and 310 therewithin for operative connection with a portion of the circuit and switch assembly 116. The sockets 308 and 310 communicate with the interior volume 300 of the barrel 150.

The interior volume 300 of the barrel 150 preferably encloses a first piston assembly 320, a second piston assembly 330 and a pressure generating element 340, which is configured to be disposed between the barrel 150 and the first piston assembly 320. The pressure generating element 340 is also termed as fluid pressure generator.

It is a particular feature of an embodiment of the present invention that the second piston assembly 330 has a hollow longitudinal actuating element 342 generally forwardly extending therefrom along longitudinal axis 344 and configured for both operatively engaging the circuit and switch assembly 116 and for enabling bi-directional passage of gas therethrough, specifically, the passage of air is enabled through the actuating element 342.

It is noted that first piston assembly 320, second piston assembly 330 and pressure generating element 340 form at least a portion of the pressure generating assembly 160 and it is particularly seen in FIGS. 7A-7C that they are mutually arranged along longitudinal axis 344 and generally symmetrical thereabout, whereas longitudinal axis 344 is preferably parallel to longitudinal axis 101.

It is noted that lid 152 is preferably coaxially arranged with the pressure generating assembly 160 and includes a central opening 346 for insertion of the actuating element 342 therethrough. The lid 152 further includes a flow path housing portion 348 adapted for receiving at least a portion of the flow path septum 120 therein.

Pressure generating element 340 in accordance with an embodiment of the present invention is a hydrogen cell, such as Cat. Number V150H2MF, commercially available from Varta, Ellwangen, Germany. Alternatively, the pressure generating element 340 can be a compressed gas reservoir or any other suitable pressure generating element.

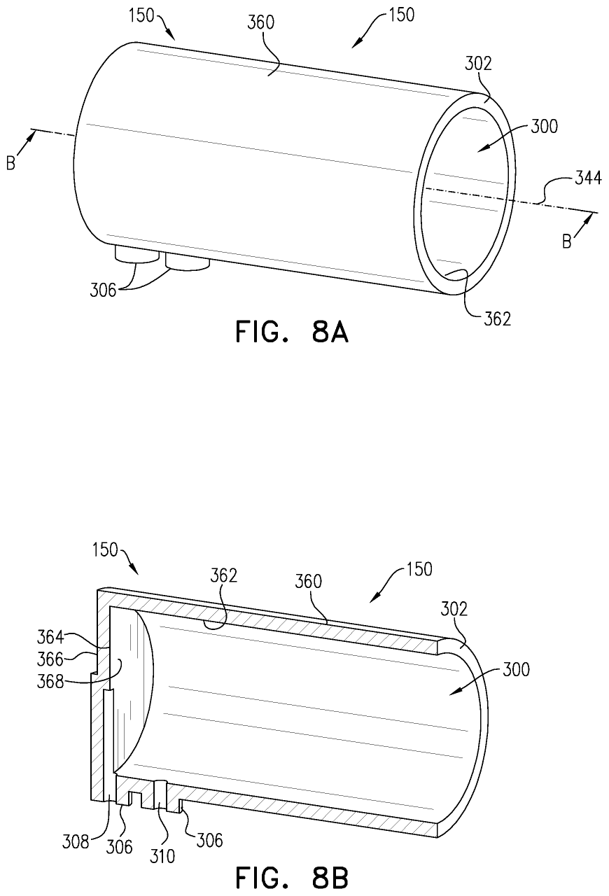

Reference is now made to FIGS. 8A and 8B, which are respective simplified pictorial illustration and sectional view of the barrel 150, forming part of the reservoir assembly 114 of FIGS. 6-7C, section being taken along lines B-B in FIG. 8A.

It is seen in FIGS. 8A & 8B that the barrel 150 is preferably an integrally made element typically having a generally circular cross-section, which is preferably made of polypropylene or any other bio-compatible material that does not harm the medicament that is adapted to be contained within the interior volume 300 of the barrel 150. The barrel 150 is generally arranged along the longitudinal axis 344. Barrel 150 defines an outer surface 360 and an inner surface 362. The barrel 150 has a rearward closed end wall 364 defining a rearwardly facing surface 366 and a forwardly facing surface 368. As mentioned above, the barrel 150 also has an open forward end defining circumferential end 302.

As also mentioned above, the barrel 150 further includes typically two protrusions 306, each having respective internal socket 308 and 310 therewithin for operative connection with a portion of the circuit and switch assembly 116. The sockets 308 and 310 communicate with the interior volume 300 of the barrel 150.

It is noted that the barrel 150 can alternatively have an oval cross-section which allows for optimal space utilization within interior volume 113 defined between the top housing portion 110 and bottom housing portion 112 and contributes to pre-defined positioning of the barrel 150 within interior volume 113.

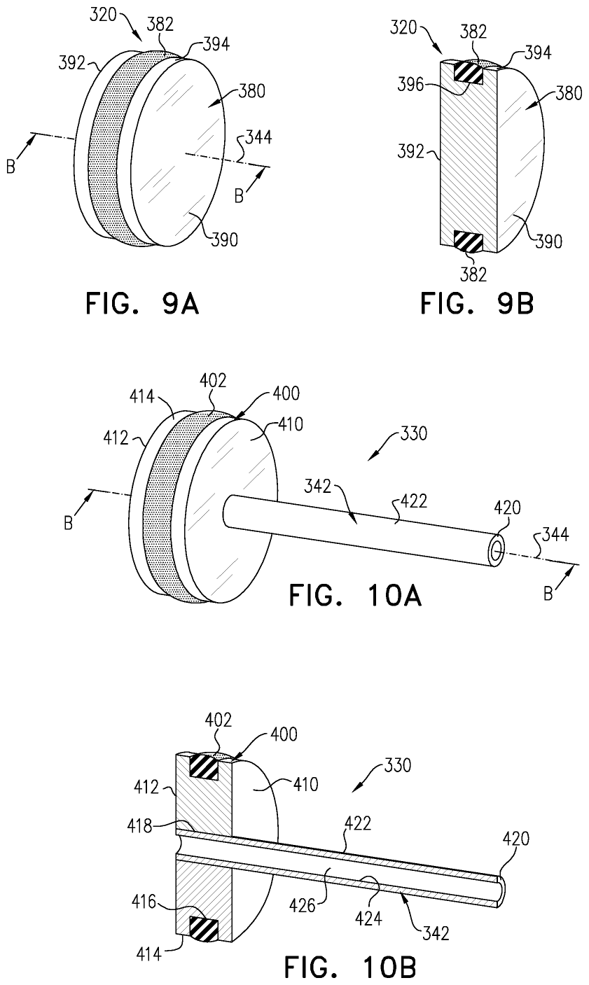

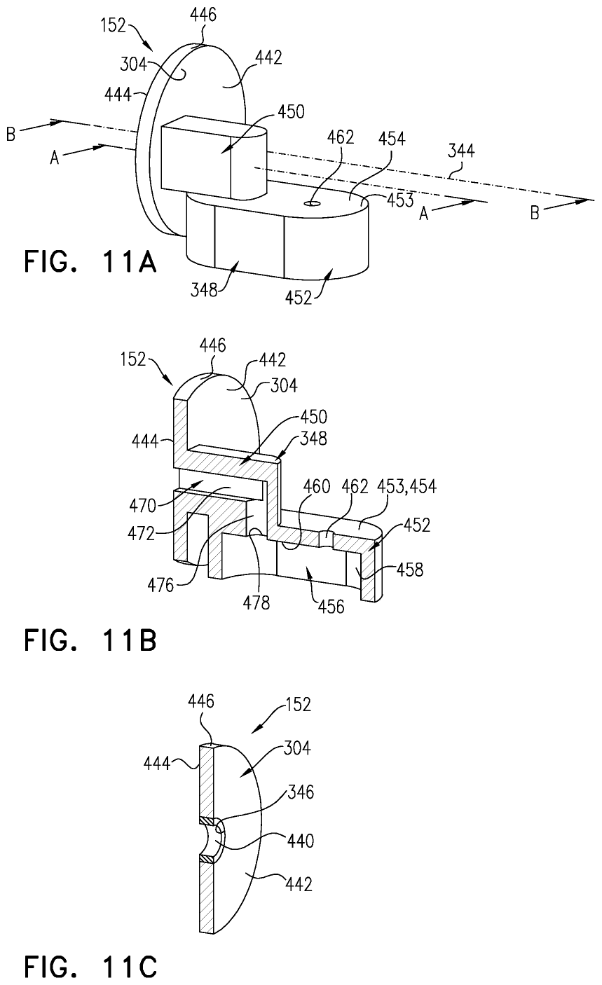

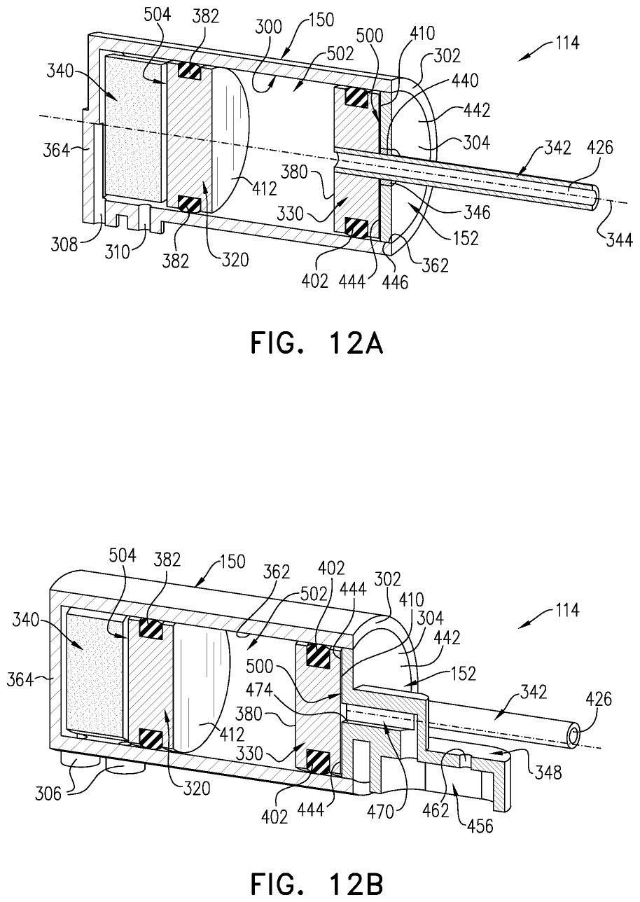

Reference is now made to FIGS. 9A and 9B, which are respective simplified pictorial illustration and sectional view of the first piston assembly 320, forming part of the reservoir assembly 114 of FIGS. 6-7C, section being taken along lines B-B in FIG. 9A.

First piston assembly 320 preferably includes a first piston 380 and a sealing ring 382, which is fixedly mounted thereon. Alternatively, the sealing ring 382 may be overmolded with the first piston 380. First piston 380 is preferably made of bio-compatible material, such as polypropylene and the sealing ring 382 is preferably made of a relatively resilient material, such as silicon. The first piston 380 preferably has a cross-section which is suitable for a tight-fit insertion within the inner volume 300 of the barrel 150 and is generally arranged along longitudinal axis 344. In this particular embodiment, the first piston 380 is disc-shaped and having a circular cross-section.

The first piston 380 defines a forwardly facing engagement surface 390 and a rearwardly facing engagement surface 392. A circumferential wall portion 394 extends between the forwardly facing engagement surface 390 and the rearwardly facing engagement surface 392. A groove 396 is formed within wall portion 394 and the sealing ring 382 is at least partially seated therewithin. The sealing ring 382 preferably slightly protrudes radially outwardly from circumferential wall portion 394.

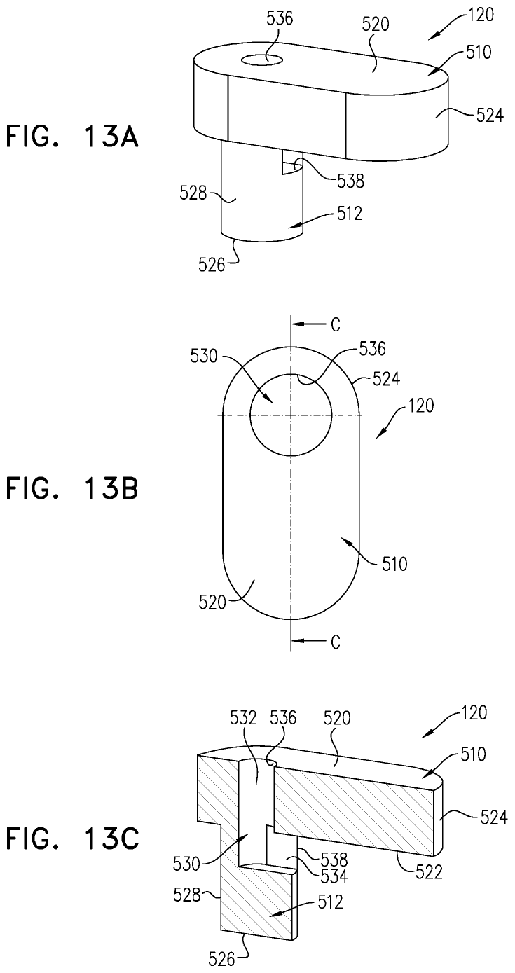

Reference is now made to FIGS. 10A and 10B, which are respective simplified pictorial illustration and sectional view of the second piston assembly 330, forming part of the reservoir assembly 114 of FIGS. 6-7C, section being taken along lines B-B in FIG. 10A.

Second piston assembly 330 preferably includes a second piston 400 and a sealing ring 402, which is fixedly mounted thereon. Alternatively, the sealing ring 402 may be overmolded with the first piston 400. Second piston 400 is preferably made of bio-compatible material, such as polypropylene and the sealing ring 402 is preferably made of a relatively resilient material, such as silicon. The second piston 400 preferably has a cross-section which is suitable for a tight-fit insertion within the inner volume 300 of the barrel 150 and is generally arranged along longitudinal axis 344. In this particular embodiment the first piston 400 is disc-shaped and having a circular cross-section.

The second piston 400 defines a forwardly facing engagement surface 410 and a rearwardly facing engagement surface 412. A circumferential wall portion 414 extends between the forwardly facing engagement surface 410 and the rearwardly facing engagement surface 412. A groove 416 is formed within wall portion 414 and the sealing ring 402 is at least partially seated therewithin. The sealing ring 402 preferably slightly protrudes radially outwardly from circumferential wall portion 414.

A central bore 418 is formed preferably at the center of the second piston 400 and extends generally longitudinally along axis 344 from the forwardly facing surface 410 to the rearwardly facing surface 412.

As mentioned above, hollow actuating element 342 generally forwardly extends from the second piston 400 along longitudinal axis 344 and configured for both operatively engaging the circuit and switch assembly 116 and for enabling bi-directional passage of gas therethrough.

The actuating element 342 is preferably made of a conductive material, such as metal for example and is either inserted into the central bore 418 of the second piston assembly 330 in a sealing engagement therewith or is integrally made with the second piston assembly 330, such as by way of insert molding, for example.

The actuating element 342 is sealingly fitted within central bore 418 and extends from rearwardly facing surface 412 to forwardly facing surface 410 and protrudes forwardly therefrom to a forwardly facing generally annular end surface 420. The actuating element 342 has an outer surface 422 and an inner surface 424 defined by a through bore 426 formed within the actuating element 342.

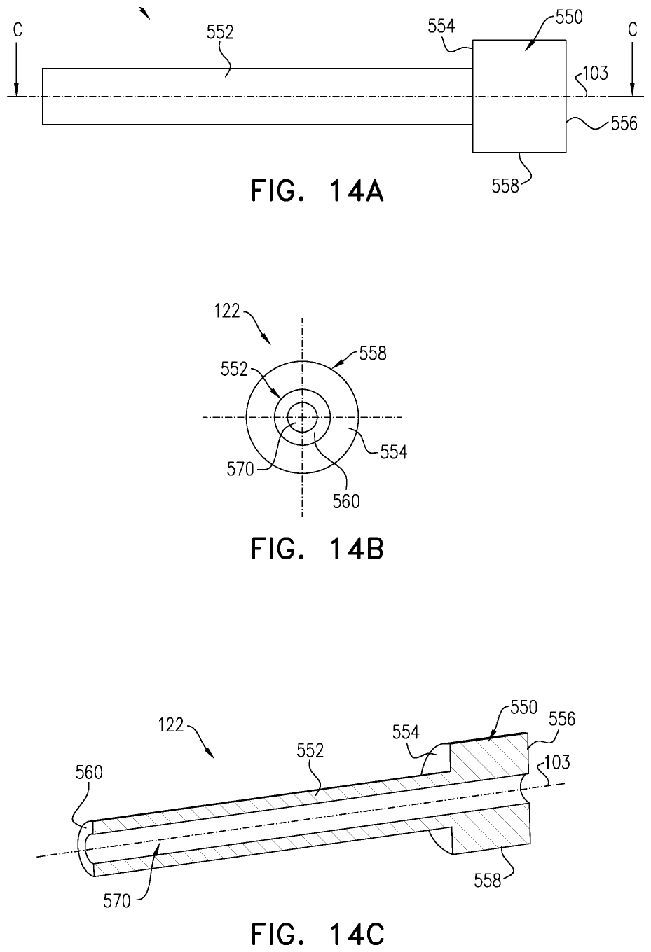

Reference is now made to FIGS. 11A-11C, which are respective simplified pictorial illustration and two sectional views of the reservoir lid 152, forming part of the reservoir assembly 114 of FIGS. 6-7C, sections being taken along lines B-B and C-C in FIG. 11A.

As mentioned above, the reservoir lid 152 has the generally circular flange 304 having central opening 346. An O-ring 440 is fixedly attached to the surface defined by central opening 346 or is integrally formed with the reservoir lid 152 by way of overmolding, for example.

The circular flange 304 has a forwardly facing surface 442, a rearwardly facing surface 444 and a circumferential generally circular wall portion 446.

As also mentioned above, the reservoir lid 152 further includes flow path housing portion 348 extending generally forwardly from flange 304. The flow path housing portion 348 has an upper portion 450 and a generally obround bottom portion 452 connected therewith.

The bottom portion 452 of the flow path housing portion 348 has a top wall portion 453 having an upwardly facing surface 454 and includes an internal socket 456, having a circumferential wall 458 and a downwardly facing surface 460. An aperture 462 is formed through the top wall portion 453 and communicates with the internal socket 456.

The upper portion 450 of the flow path housing portion 348 includes an internal fluid pathway 470 having a first pathway portion 472 terminating at an opening 474 formed in flange 304 and connecting with a second pathway portion 476 terminating at an opening 478 formed in top wall portion 453 and communicating with internal socket 456. It is seen particularly in FIG. 11B that the second pathway portion 476 is preferably disposed transversely to the first pathway portion 472.

It is noted that opening 474 is radially offset from the central opening 346 of the reservoir lid 152.

Reference is now made to FIGS. 12A and 12B, which are two simplified sectional views of the reservoir assembly 114 of FIG. 6, sections being taken along lines A-A and B-B in FIG. 6.

It is noted that the reservoir assembly 114 is shown in an initial storage orientation in FIGS. 12A & 12B.

It is seen in FIGS. 12A & 12B that the reservoir lid 152 is fixedly attached to the barrel 150, such as by heat welding for example, so that the circumferential wall 446 of the reservoir lid 152 abuts the inner surface 362 of barrel 150 and forwardly facing surface 442 of the lid 152 is generally aligned with circumferential edge 302.

It is further seen in FIGS. 12A & 12B that the pressure generating element 340, first piston assembly 320 and a portion of the second piston assembly 330 are all enclosed within the interior volume 300 of the barrel 150, which is confined at its forward end by reservoir lid 152.

The pressure generating element 340 is generally statically disposed at the rearward end of the barrel 150, adjacent rearward closed end wall 364 of the barrel 150 and communicating with internal sockets 308 and 310 of the barrel 150.

The first piston assembly 320 is preferably sealingly slidably disposed within the interior volume 300 of barrel 150, forwardly of the pressure generating element 340, preferably in close proximity thereto. The sealing element 382 of the first piston assembly 320 sealingly engages the inner surface 362 of the barrel 150.

It is a particular feature of an embodiment of the present invention that the second piston assembly 330 is preferably sealingly slidably disposed within the interior volume 300 of barrel 150, adjacent the forward end of the barrel 150, such that forwardly facing engagement surface 410 of the second piston assembly 330 abuts rearwardly facing surface 444 of flange 304 of the reservoir lid 152. The sealing element 402 of the second piston assembly 330 sealingly engages the inner surface 362 of the barrel 150.

It is a further particular feature of an embodiment of the present invention that the actuating element 342 extends through central opening 346 of the reservoir lid 152 and protrudes forwardly of the forwardly facing surface 442 of the reservoir lid 152. O-ring 440 of the lid 152 is configured to seal around the actuating element 342.

It is a particular feature of an embodiment of the present invention that in this initial orientation, a medication volume 500 is defined between the second piston assembly 330 and the reservoir lid 152 and substantially equals zero in this initial orientation. The medication volume 500 is adapted to communicate with internal fluid pathway 470 of the flow path housing portion 348. A gas volume 502 is defined between the first piston assembly 320 and the second piston assembly 330, which are preferably longitudinally spaced from each other along longitudinal axis 344. The gas volume 502 preferably contains air in this initial orientation and is adapted to communicate with the atmosphere through bore 426 formed in the actuating element 342. A pressure chamber 504 is defined between the first piston assembly 320 and the pressure generating element 340 and this pressure chamber 504 is fluidly sealed by means of sealing engagement between sealing element 382 of the first piston assembly 320 and the inner surface 362 of the barrel 150, and is configured for allowing pressure build-up within pressure chamber 504 for slidably displacing the first piston assembly 320 axially forwardly toward reservoir lid 152. Medication volume 500, gas volume 502 and pressure chamber 504 are all variable volumes, which form part of the interior volume 300 of the barrel 150.

Reference is now made to FIGS. 13A-13C, which are respective simplified pictorial illustration, top view and a sectional view of the flow path septum 120, forming part of the transfusion pump 100 of FIG. 1, section being taken along lines C-C in FIG. 13B.

Flow path septum 120 is an integrally formed element, generally made of a resilient material, such as silicon for example. The flow path septum 120 includes a generally obround base portion 510 and a generally cylindrical protrusion 512 extending therefrom.

The base portion 510 defines an upwardly facing surface 520, a downwardly facing surface 522 and a circumferential wall 524.

The cylindrical protrusion 512 extends downwardly from the downwardly facing surface 522 and terminates at a downwardly facing end 526. The protrusion 512 defines a circumferential wall 528.

A fluid flow path 530 is formed within flow path septum 120 and includes a first fluid flow path portion 532 and a second fluid flow path portion 534, generally transversely extending with respect to each other.

The first fluid flow path portion 532 extends axially downwardly from upwardly facing surface 520, forming an opening 536 therein. The second fluid flow path portion 534 extends from the first fluid flow path portion 532 and disposed generally transversely thereto. The second fluid flow path portion 534 generally extends through protrusion 512 and defines an opening 538 in the circumferential wall 528 thereof.

Reference is now made to FIGS. 14A-14C, which are respective simplified pictorial illustration, top view and a sectional view of the cannula 122, forming part of the transfusion pump 100 of FIG. 1, section being taken along lines C-C in FIG. 14A.

Cannula 122 is an integrally formed element generally made of a resilient material, such as silicon and arranged along longitudinal axis 103.

It is seen in FIGS. 14A-14C that the cannula 122 has a base portion 550 and a hollow generally cylindrical portion 552 extending axially downwardly therefrom, and defining a downwardly facing shoulder 554 therebetween. The base portion 550 has an upwardly facing surface 556 and a circumferential wall 558. The cylindrical portion 552 defines a downwardly facing edge 560. A through bore 570 is formed through the entire cannula 122 and extends through both the cylindrical portion 552 and the base portion 550.

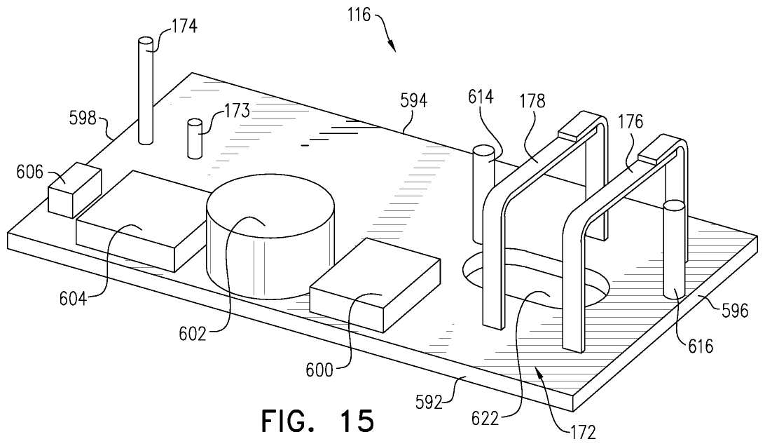

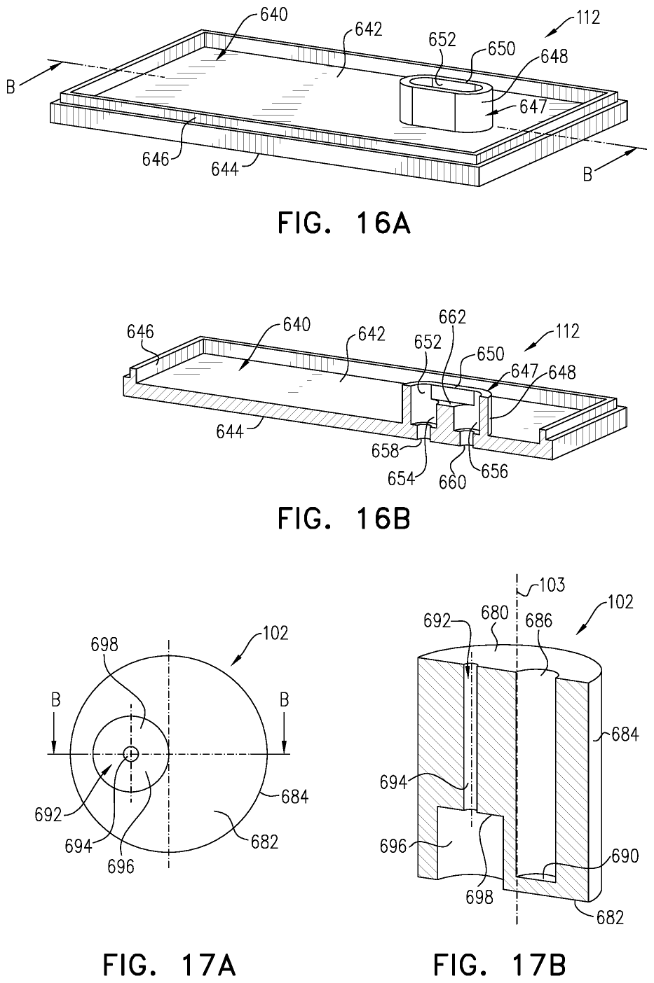

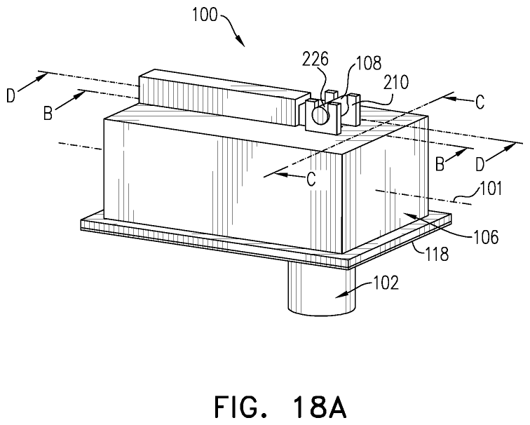

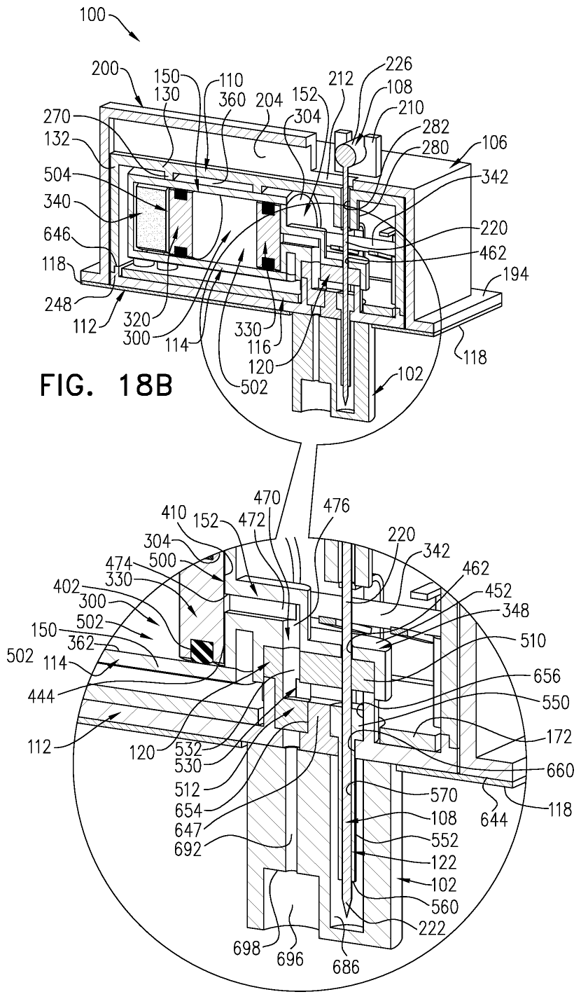

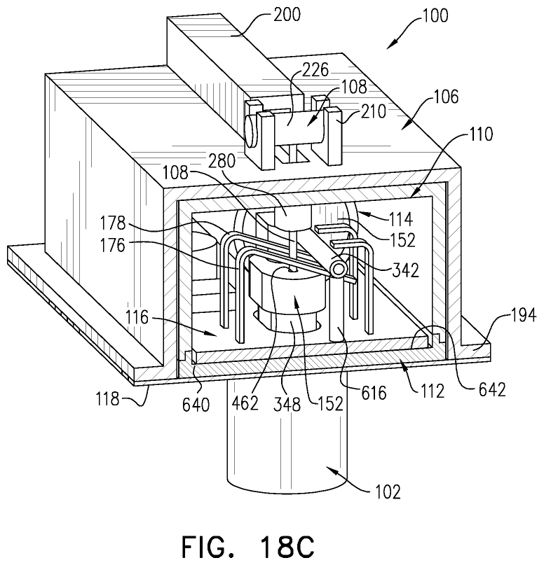

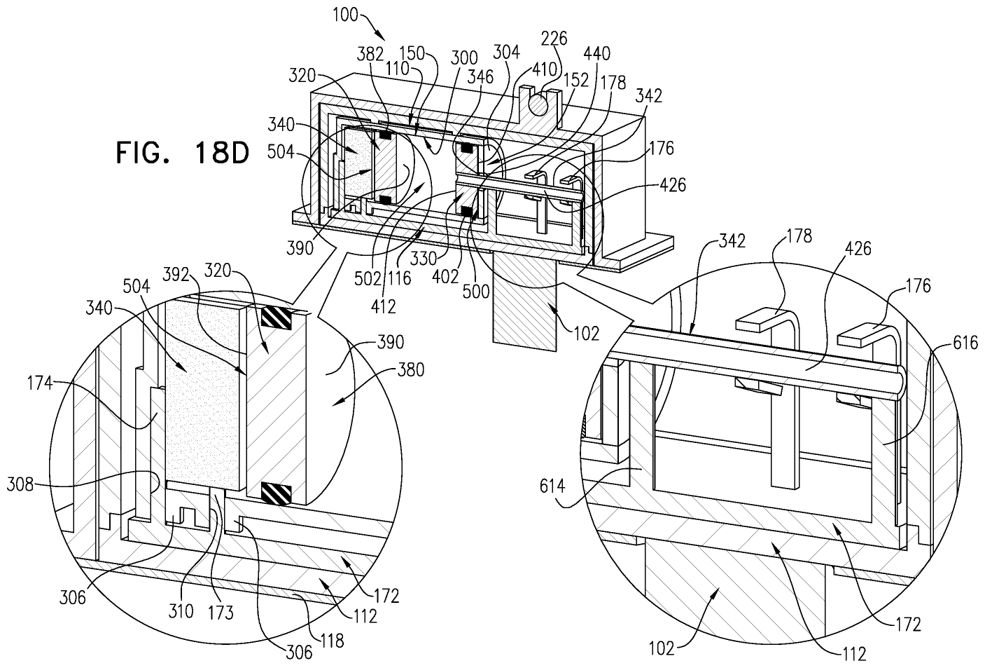

Reference is now made to FIG. 15, which is a simplified pictorial illustration of the circuit and switch assembly 116, forming part of the transfusion pump 100 of FIG. 1.

As mentioned above, the circuit and switch assembly 116 is made of printed circuit board 172. The rigid printed circuit board 172 defines a first side edge 592, a second side edge 594, a front edge 596 and a rear edge 598. The printed circuit board 172 has various electrical components preferably formed thereon, such as a CPU 600, battery 602 adapted to provide electricity to various components of the transfusion pump 100, a buzzer 604 and a LED 606. Various capacitors or resistors may be formed on printed circuit board 172 as well.

Typically, two electrical contacts 173 and 174, preferably in a form of actuating elements extend generally upwardly from the printed circuit board 172 and are operative for electrical coupling with at least a portion of the pressure generating element 340, which is enclosed within the reservoir assembly 114. The two electrical contacts 173 and 174 are preferably disposed adjacent the rear edge 598.

Typically, two electrical contacts 614 and 616, preferably in a form of actuating elements, are formed generally adjacent the front edge 596 and serve as switches.

As also mentioned above and seen in FIG. 15, generally two axially spaced switches 176 and 178 are disposed between the two electrical contacts 614 and 616. It is seen that the switches 176 and 178 are shown in a closed operative orientation in this illustration.

An aperture 622 is formed in the circuit board 172, adapted for insertion of at least a portion of the flow path housing portion 348 of the reservoir lid 152 therethrough.

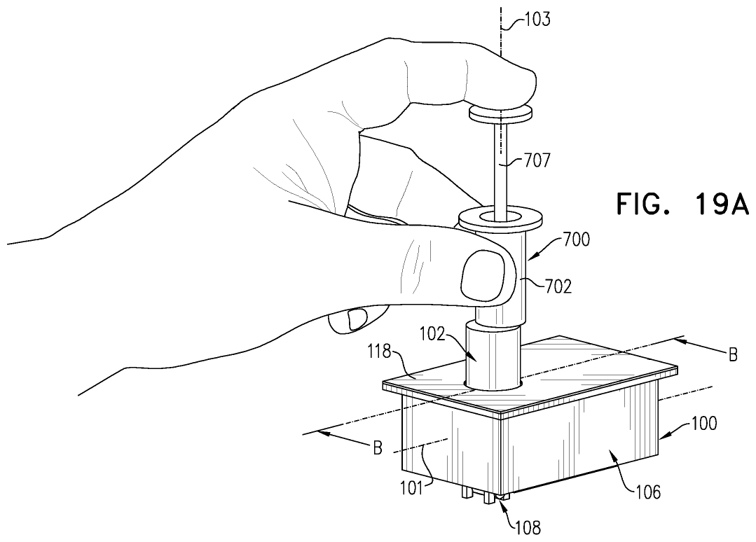

Reference is now made to FIGS. 16A and 16B, which are respective simplified pictorial illustration and sectional view of the bottom housing portion 112 of the transfusion pump 100 of FIG. 1, section being taken along lines B-B in FIG. 16A.

Bottom housing portion 112 is an integrally formed element, preferably made of plastic, having a flat base wall 640 defining an upwardly facing surface 642, a downwardly facing surface 644 and a generally circumferential rim 646 extending generally upwardly from the upwardly facing surface 642 thereof and adapted to fit the corresponding portion of top housing portion 110.

An upwardly extending protrusion 647 is formed on the flat base wall 640 and extends generally upwardly therefrom. The protrusion 647 has a generally obround circumferential wall portion 648 defining an upwardly facing edge 650.

The upwardly extending protrusion 647 defines an inner surface 652 and typically two generally spaced openings 654 and 656 each forming a respective aperture 658 and 660 in base wall 640 and a recess 662 which generally joins the two openings 654 and 656 and is disposed preferably adjacent the upwardly facing edge 650.

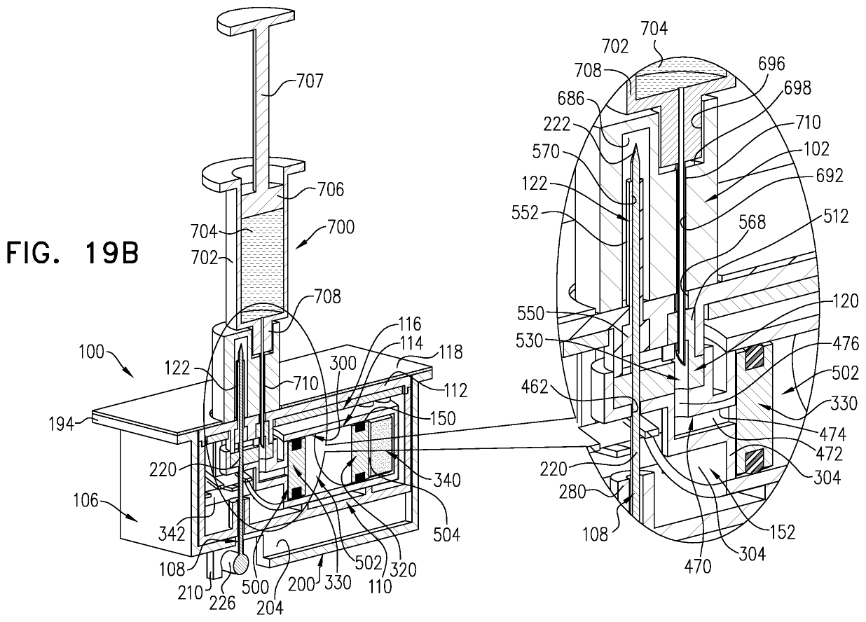

Reference is now made to FIGS. 17A and 17B, which are respective simplified top view and pictorial sectional view illustrations of the filling port 102 associated with the transfusion pump 100 of FIG. 1, section being taken along lines B-B in FIG. 17A.

The filling port 102 is an integrally formed element preferably made of plastic and arranged along axis 103.

It is noted that the filling port 102 is adapted to be removably mounted to the transfusion pump 100.

The filling port 102 is a generally cylindrical element having an upwardly facing wall 680, a downwardly facing wall 682 and a circumferential surface 684. A first eccentric opening 686 is formed in the filling port 102 and extends downwardly from the upwardly facing wall 680, terminating at an upwardly facing surface 690. A second eccentric opening 692 is formed in the filling port 102 and extends through the entire filling port 102. The second eccentric opening 692 includes a first portion 694 having a first diameter and extending from the upwardly facing wall 680 to a location preferably generally adjacent the downwardly facing wall 682. The second eccentric opening 692 further includes a second portion 696 having a second diameter, generally greater than the first diameter and extending from the downwardly facing wall 682 upwardly to the same location generally adjacent the downwardly facing wall 682. A downwardly facing shoulder 698 is formed between the first portion 694 and the second portion 696.

Reference is now made to FIGS. 18A-18D, which are respective simplified pictorial illustration and three sectional views of the transfusion pump 100 of FIG. 1 shown in a first operative orientation, sections being taken along lines B-B, C-C and D-D in FIG. 18A.

The transfusion pump 100 as illustrated in FIGS. 18A-18D is in a storage operative orientation.

It is seen in FIGS. 18A-18D that the reservoir assembly 114 is disposed in its initial operative orientation, as described with reference to FIGS. 12A & 12B.

Bottom housing portion 112 is generally attached to top housing portion 110, such that downwardly facing shoulder 248 of the top housing portion 110 is fixedly attached to circumferential rim 646 of the bottom housing portion 112, such as by way of heat welding. Adhesive layer 118 is fixedly attached to the downwardly facing surface 644 of the bottom housing portion 112.

The reservoir assembly 114, which is disposed in its initial operative orientation as described in FIGS. 12A & 12B is fixedly fitted within the interior volume 113 enclosed between the top housing portion 110 and the bottom housing portion 112. Outer surface 360 of the barrel 150 of reservoir assembly 114 is supported by arcuate protrusions 270 of top housing portion 110.

Circuit and switch assembly 116 is supported on the upwardly facing surface 642 of the bottom housing portion 112, disposed between the bottom housing portion 112 and the reservoir assembly 114. It is specifically seen in FIG. 18D that electrical contacts 173 and 174 of the circuit and switch assembly 116 extend through respective internal sockets 310 and 308 in the barrel 150 for operative electrical coupling with the pressure generating element 340 upon receiving an appropriate command from the CPU 600.

It is a particular feature of an embodiment of the present invention that in this storage operative orientation, the medication volume 500 within barrel 150 substantially equals zero, such that forwardly facing engagement surface 410 of the second piston assembly 330 abuts the flange 304 of reservoir lid 152. In this orientation, the actuating element 342 extends forwardly from the reservoir lid 152 to the maximal longitudinal extent. The fact that the second piston assembly 330 abuts the reservoir lid 152 in storage operative orientation obviates priming of the reservoir assembly 114.

It is a further particular feature of an embodiment of the present invention, as is seen particularly in FIG. 18D, that when the actuating element 342 extends forwardly from the reservoir lid 152 to its maximal longitudinal extent, it mechanically disconnects the pressure generating element 340 from the electrical circuit of the circuit and switch assembly 116. Specifically, the actuating element 342 physically opens switches 176 and 178 of the circuit and switch assembly 116, so that the electrical circuit between the switches 176 and 178 and electrical contacts 173 and 174 is open and any current leakage to the pressure generating element 340 is prevented. Switches 176 and 178 are disposed in a non-actuated orientation in this storage operative orientation.

It is further seen specifically in FIGS. 18C & 18D that the actuating element 342 in this storage operative orientation contacts both electrical contacts 614 and 616, thus closing the electrical circuit therebetween and providing an indication to the CPU 600 that the transfusion pump 100 is empty of medication, meaning that the medication volume 500 equals zero.

As mentioned above, O-ring 440 of the reservoir lid 152 seals around the actuating element 342.

It is specifically seen in FIG. 18B that flow path septum 120 is fixedly enclosed between the flow path housing portion 348 of the reservoir lid 152 and the protrusion 647 of the bottom housing portion 112. Specifically, obround base portion 510 of the flow path septum 120 is sealingly received within obround bottom portion 452 of the reservoir lid 152 and protrusion 512 of the flow path septum 120 is sealingly received within opening 654 of the bottom housing portion 112.

A portion of the cannula 122 is sealingly seated within protrusion 647 of the bottom housing portion 112, such that base portion 550 of cannula 122 is seated within opening 656 of the bottom housing portion 112 and the cylindrical portion 552 of cannula 122 protrudes downwardly of the bottom housing portion 112 through opening 660.

It is a particular feature of an embodiment of the present invention that inserter 106 is removably mounted onto the transfusion pump 100 in this storage operative orientation, such that the top housing portion 110 is enclosed within the inserter 106 and the circumferential flange 194 of the inserter 106 is generally aligned with the base wall 640 of the bottom housing portion 112.

It is a particular feature of an embodiment of the present invention that the penetrating element 108 is pivotably held by the inserter 106 and is biased to a retracted position, as described in detail hereinbelow. The penetrating element 108 is disposed in an extended position in this storage operative orientation and is pivotably coupled to the inserter 106 by means of insertion of axle 226 of the penetrating element 108 into hooks 210 of the inserter 106.

It is particularly seen in FIG. 18B that the longitudinal portion 220 of the penetrating element 108 extends through opening 212 in the inserter 106, via through bore 282 of top housing portion 110, further extends through aperture 462 of reservoir lid 152, penetrates the base portion 510 of the flow path septum 120 and into the through bore 570 of the cannula 122, while the sharp end 222 of the penetrating element 108 generally protrudes downwardly from downwardly facing edge 560 of the cannula 122.

It is seen in FIG. 18B that fluid pathway 470 of the reservoir lid 152 and fluid flow path 530 of the flow path septum 120 are aligned in this storage operative orientation, however fluid passage into the cannula 122 is prevented due to the sealable insertion of the penetrating element 108 into the through bore 570 of the cannula 122. It is noted that fluid pathway 470 of the reservoir lid 152 is adapted to communicate with the medication volume 500 through opening 474 of the reservoir lid 152.

Filling port 102 is removably mounted onto the transfusion pump 100 in this storage operative orientation. The filling port 102 serves both for attachment of a pre-filled syringe (not shown) thereto to fill the transfusion pump 100 with medication and for protecting the cannula 122 and the penetrating element 108, which protrude downwardly from the bottom housing portion 112.

It is specifically seen that the longitudinal portion 552 of cannula 122 with a portion of the penetrating element 108 extending therethrough are located within opening 686 of the filling port 102 and are protected therewithin. Second eccentric opening 692 of the filling port 102 is adapted for insertion of a needle of the pre-filled syringe thereinto. The fluid flow path 530 of the flow path septum 120 is sealed by protrusion 512, thus preventing communication thereof with second eccentric opening 692 of the filling port 102 in this storage operative orientation.

It is noted that in this storage operative orientation, all portions of the transfusion pump 100 which contact the medication have to be sterilized, preferably by ETO.

It is a particular feature of an embodiment of the present invention that the medication volume 500 and flow path 530 of the flow path septum 120 and flow path 470 of the reservoir lid 152 are sterilized by passage of ETO through opening 692 of the filling port 102, further through flow path septum 120, through flow path 530 of the flow path septum 120, into flow path 470 of the reservoir lid 152 and finally into the medication volume 500 defined by the forwardly facing engagement surface 410 of the second piston assembly 330, sealing element 402, inner surface 362 of the barrel 150 and rearwardly facing surface 444 of the reservoir lid 152.

It is noted that alternatively, the penetrating element 108 may be formed as a hollow needle having an opening at its side, provided between the base portion 550 of the cannula 122 and the base portion 510 of the flow path septum 120, thus providing a fluid flow passage for the ETO through the penetrating element 108 into flow path 530 of the flow path septum 120, further into flow path 470 of the reservoir lid 152 and finally into the medication volume 500.

It is a particular feature of an embodiment of the present invention that gas volume 502 disposed between the first piston assembly 320 and the second piston assembly 330 can be sterilized by a sterilizing agent, such as ETO, which passes through the bore 426 of the actuating element 342. The inner volume 113 enclosed between the top housing portion 110 and the bottom housing portion 112 is not sealed, thus passage of ETO therethrough is enabled, ETO passes through bore 426 of the actuating element 342 into gas volume 502 defined by the rearwardly facing engagement surface 412 of the second piston assembly 330, sealing element 402, inner surface 362 of the barrel 150, forwardly facing engagement surface 390 of the first piston assembly 320 and sealing element 382.

It is noted that the pressure chamber 504, which is defined between the first piston assembly 320 and the pressure generating chamber 340, is minimal and preferably equals zero in this storage operative orientation. The volume of the pressure chamber 504 is sealed by means of sealing element 382, which forms part of the first piston assembly 320.

It is a particular feature of an embodiment of the present invention that the transfusion pump 100 has a barrel 150 enclosed between top housing portion 110 and bottom housing portion 112, the barrel 150 contains the variable medication volume 500 configured to contain medication 704 (not shown) upon filling of the barrel 150, the variable gas volume 502 configured to contain air before filling of the barrel 150 and the pressure chamber 504 configured to contain the pressure generating element 340. The medication volume 500, gas volume 502 and pressure chamber 504 are fluidly sealed with respect to each other and the medication volume 500 is selectably fluidly couplable with the cannula 122 and the gas volume 502 is fluidly couplable with the atmosphere.

It is a further particular feature of an embodiment of the present invention that the medication volume 500 is mutually variable with the gas volume 502 during filling of the medication volume 500 with medication 704, such that the volume increase of the medication volume 500 corresponds to volume decrease of the gas volume 502, such that pressure build-up in the gas chamber 502 is prevented.

It is a still further particular feature of an embodiment of the present invention that the medication volume 500 is mutually variable with the volume of the pressure chamber 504 during pumping of the medication 704 out of the medication volume 500 through the cannula 122. Firstly, the volume increase of the pressure chamber 504 corresponds to the volume decrease of the gas chamber 502, up to engagement of the first piston assembly 320 and the second piston assembly 330. Secondly, the volume increase of the pressure chamber 504 corresponds to volume decrease of the medication volume 500 upon further displacement of the two piston assemblies 320 and 330 together in a medicament pumping direction.

Reference is now made to FIGS. 19A and 19B, which are respective simplified pictorial illustration and sectional view of the transfusion pump 100 of FIG. 1 shown in a second operative orientation, section being taken along lines B-B in FIG. 19A.

The transfusion pump 100 is shown in FIGS. 19A & 19B in a syringe attachment operative orientation.

It is seen in FIGS. 19A & 19B that the transfusion pump 100 is turned upside down in comparison with the orientation illustrated and described with reference to FIGS. 18A-18D, so that the filling port 102 now protrudes upwardly from the bottom housing portion 112 along axis 103 and a pre-filled syringe 700 is attached thereto. The pre-filled syringe 700 includes a syringe barrel 702 containing a medication 704 confined by a piston 706 and a plunger rod 707 fixedly attached to the piston 706. A luer 708 extends from the syringe barrel 702 and a needle 710 is preferably fixedly attached thereto.

In this syringe attachment operative orientation, it is seen that the luer 708 of the syringe 700 is inserted into the second portion 696 of opening 692 of the filling port 102 up to engagement of the luer 708 with shoulder 698 of the filling port 102. The needle 710 of the syringe 700 is inserted through opening 692 of the filling port 102 and pierces the protrusion 512 of the flow path septum 120, such that the needle 710 protrudes into first fluid flow path portion 532 of fluid flow path 530 of the flow path septum 120, thereby establishing a fluid flow passage defined between the needle 710, the first fluid flow path portion 532 of fluid flow path 530, via second pathway portion 476 and through the first pathway portion 472 of fluid pathway 470 of the reservoir lid 152 and into medication volume 500 through opening 474 of the reservoir lid 152.

It is seen that in this operative orientation illustrated in FIGS. 19A & 19B the medication 704 is still contained within the syringe 700 and thus the medication volume 500 is still entirely empty of fluid before filling thereof, due to the fact that the second piston assembly 330 abuts the flange 304 of the reservoir lid 152, thus obviating the need for priming of barrel 150 and avoiding any residual air bubbles that may have otherwise remain within the medication volume 500 after filling of medication 704. It is noted that the gas volume 502, disposed between the first piston assembly 320 and the second piston assembly 330 is generally filled with air in this operative orientation.

It is appreciated that all remaining spatial relationships between the various components of the transfusion pump 100 as described with reference to FIGS. 18A-18D generally remain unchanged in this second operative orientation.

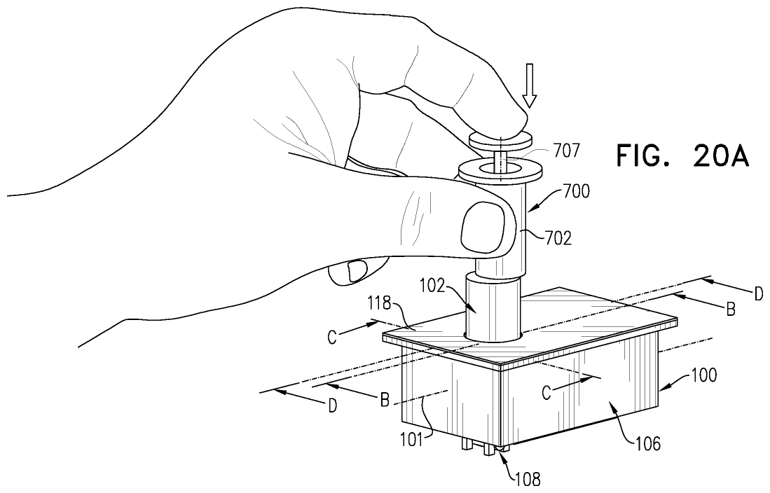

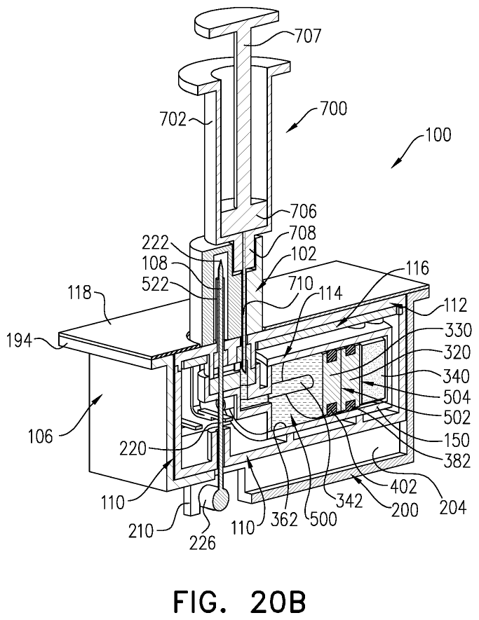

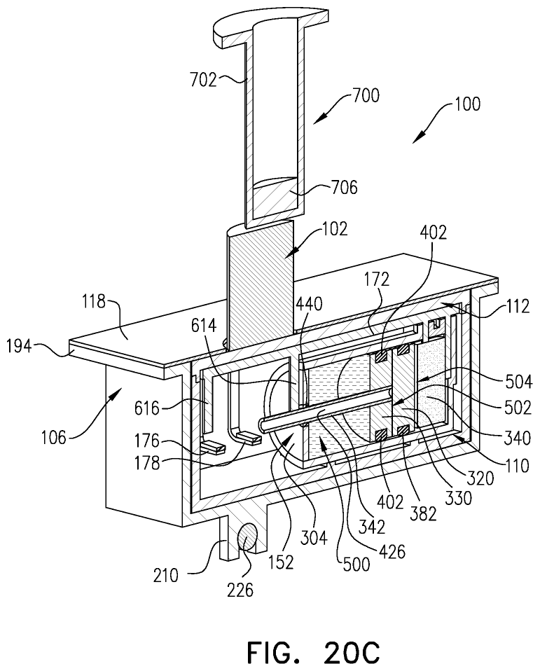

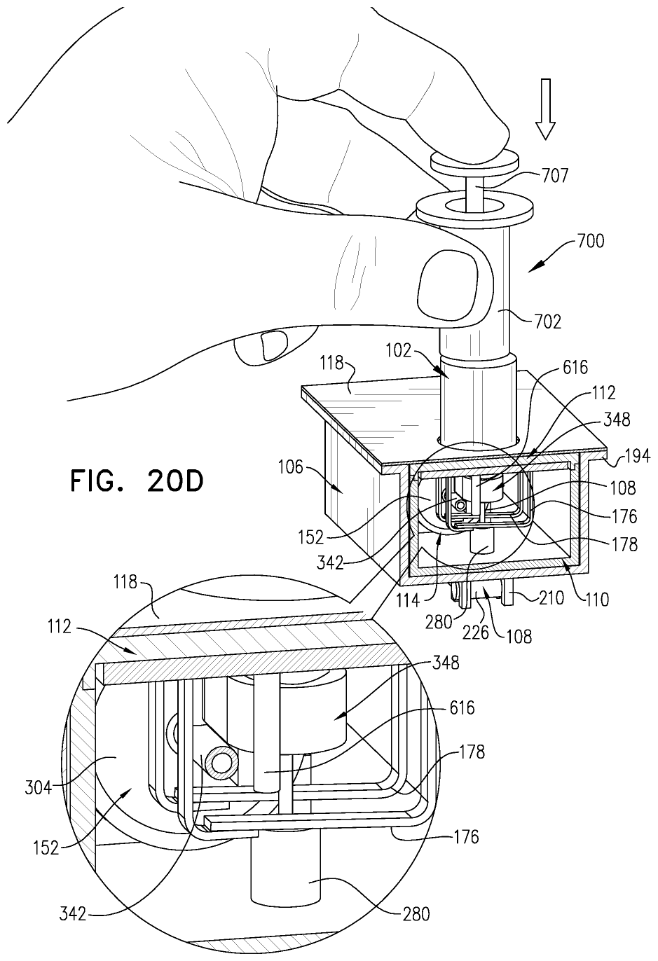

Reference is now made to FIGS. 20A-20D, which are respective simplified pictorial illustration and three sectional views of the transfusion pump 100 of FIG. 1 shown in a third operative orientation, sections being taken along lines B-B, C-C and D-D in FIG. 20A.

The transfusion pump 100 is shown in FIGS. 20A-20D in a filling operative orientation.

In this filling orientation, it is seen that the plunger 707 of the syringe 700 is pushed generally downwardly to expel at least a portion of the medication 704 contained in the syringe 700 into the reservoir assembly 114, thereby producing the medication volume 500 within the barrel 150 through needle 710, via first fluid flow path portion 532 of fluid flow path 530 of the flow path septum 120, further via second pathway portion 476 and through the first pathway portion 472 of fluid pathway 470 of the reservoir lid 152 and into medication volume 500 through opening 474 of the reservoir lid 152.

It is noted that the medication 704 is sealed within medication volume 500 due to fluid-tight sealing engagement of sealing ring 402 of the second piston assembly 330 with the inner surface 362 of the barrel 150. The medication volume 500 preferably contains no air bubbles, since the medication volume 500 of the barrel 150 before filling was entirely empty of fluid.

It is seen that in this operative orientation illustrated in FIGS. 20A-20D the medication 704 is now contained within the barrel 150, thus the medication volume 500 is increased due to the fact that the second piston assembly 330 is now rearwardly axially displaced from the flange 304 of the reservoir lid 152. In this particular embodiment, it is seen that the second piston assembly 330 now abuts the first piston assembly 320, however it is appreciated that if a smaller amount of medication is filled into the medication volume 500 of the barrel 150, then the second piston assembly 330 is axially forwardly spaced from the first piston assembly 320.

It is a particular feature of an embodiment of the present invention that the gas volume 502 is now minimized, due to the fact that the air that was previously contained within gas volume 502, as illustrated in FIGS. 19A & 19B, is now released to the atmosphere through bore 426 of the actuating element 342. In this particular embodiment, it is seen that the gas volume 502 is eliminated and now substantially equals zero, however it is appreciated that if a smaller amount of medication is filled into the medication volume 500 of the barrel 150, then the gas volume 502 is decreased and the second piston assembly 330 is axially forwardly spaced from the first piston assembly 320.

It is noted that the second piston assembly 330 is axially displaced rearwardly during filling of the medication volume 500 and the first piston assembly 320 remains generally static during the filling stage to keep the volume of the pressure chamber 504 constant. It is also noted that the volume of the pressure chamber 504 remains constant in this filling operative orientation as the pressure generating element 340 is not yet actuated.

It is a particular feature of an embodiment of the present invention that the filling of the medication volume 500 is allowed due to the fact that the air is expelled from gas chamber 502 by way of passage from the gas chamber 502 to the atmosphere through the actuating element 342.

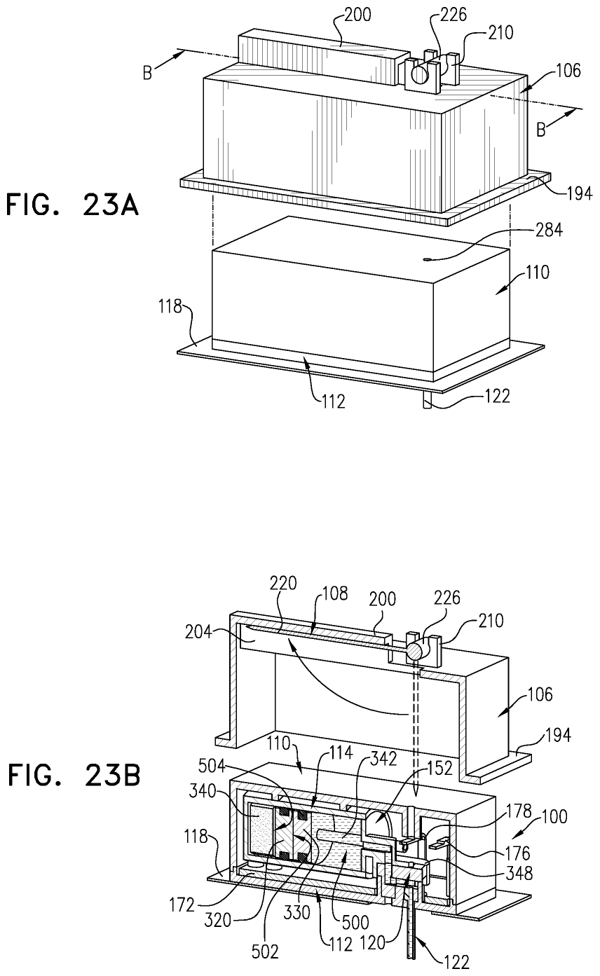

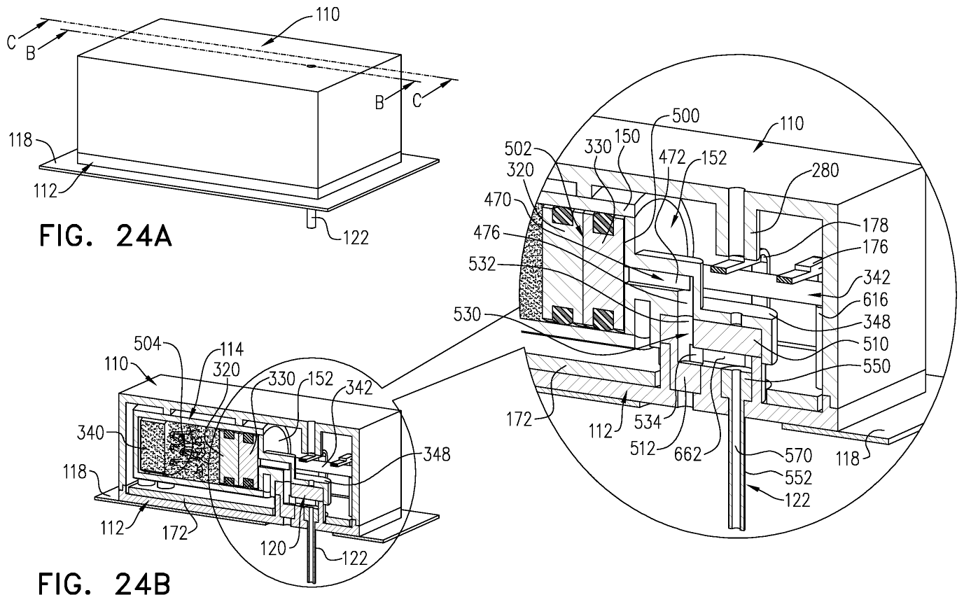

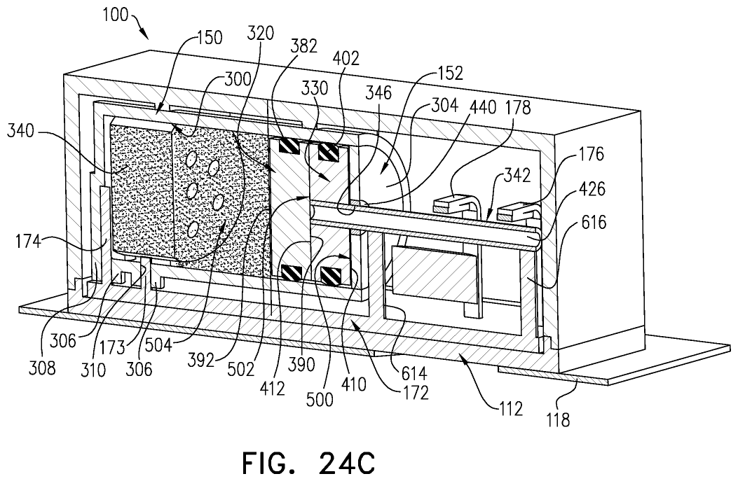

It is a further particular feature of an embodiment of the present invention that two flow paths are formed within the transfusion pump 100, which are operative separately in different operative orientations and are fluidly sealed from each other. Specifically, medication fluid path is defined by medication volume 500, fluid passageway 470 of the reservoir lid 152, flow path 530 of the flow path septum 120 and the bore 570 of the cannula 122 once the medication fluid path is established, as further described in detail with reference to FIGS. 23A-24C. Gas fluid path is defined by gas volume 502, and bore 426 of the actuating element 342. The gas fluid path and the medication fluid path are fluidly sealed from each other due to O-ring 440, which seals around the actuating element 342 and sealing ring 402 which seals between the second piston assembly 330 and the inner surface 362 of the barrel 150.

It is a particular feature of an embodiment of the present invention, as is seen particularly in FIGS. 20C & 20D, that the actuating element 342 is now rearwardly axially displaced and extends forwardly from the reservoir lid 152 to a lesser longitudinal extent than in FIGS. 18A-18D, this longitudinal extent depends on the amount of medication that is filled into the medication volume 500. It is appreciated that in this filling operative orientation, the actuating element 342 is now disposed rearwardly of both switches 176 and 178 of the circuit and switch assembly 116, thus does not physically interfere with the switches 176 and 178 and thereby causing closing thereof, and in turn causing closing of the electrical circuit. Specifically, switches 176 and 178 are now irreversibly disposed in an actuated orientation and the electrical circuit between the switches 176 and 178 and electrical contacts 173 and 174 is now closed and the pressure generating element 340 is now electrically operatively coupled with the CPU 600.

It is noted that once the electrical circuit is closed and the pressure generating element 340 is electrically coupled with the CPU 600, a timer which forms part of the circuit and switch assembly 116, counts a predetermined amount of time until the injection can be initiated, thus in this operative orientation the pressure generating element 340 is not yet actuated until a proper command is received from the CPU 600.

It is noted that in case a pre-determined minimal amount of medication is not filled into the medication volume 500, the actuating element 342 is not sufficiently rearwardly displaced, thus not placing at least one of the switches 176 and 178 in their actuated orientation. Preferably, during the filling of the medication volume 500, the actuating element 342 first stops interfering with switch 176, thus closing the electrical circuit therebetween and the electrical contacts 173 and 174 and once the minimal amount of medication is filled into the medication volume 500, the actuating element 342 also stops interfering with switch 178, thus placing both switches 176 and 178 in their actuated orientation. Both of the switches 176 and 178 need to be placed in their actuated orientation in order to actuate the pressure generating element 340.

It is a further particular feature of an embodiment of the present invention that the actuating element 342 in this filling operative orientation now contacts only electrical contact 614, but not electrical contact 616, thus opening the electrical circuit therebetween and providing an indication that the transfusion pump 100 is filled with medication.

It is a still further particular feature of an embodiment of the present invention that the actuating element 342 of the second piston assembly 330 that extends forwardly through the lid 152 is operative for electrically coupling the pressure generating element 340 to the circuit and switch assembly 116 upon driving of the at least one of the first piston assembly 320 and the second piston assembly 330 in a medication filling displacement direction.

It is appreciated that all remaining spatial relationships between the various components of the transfusion pump 100 as described with reference to FIGS. 19A & 19B generally remain unchanged in this third operative orientation.

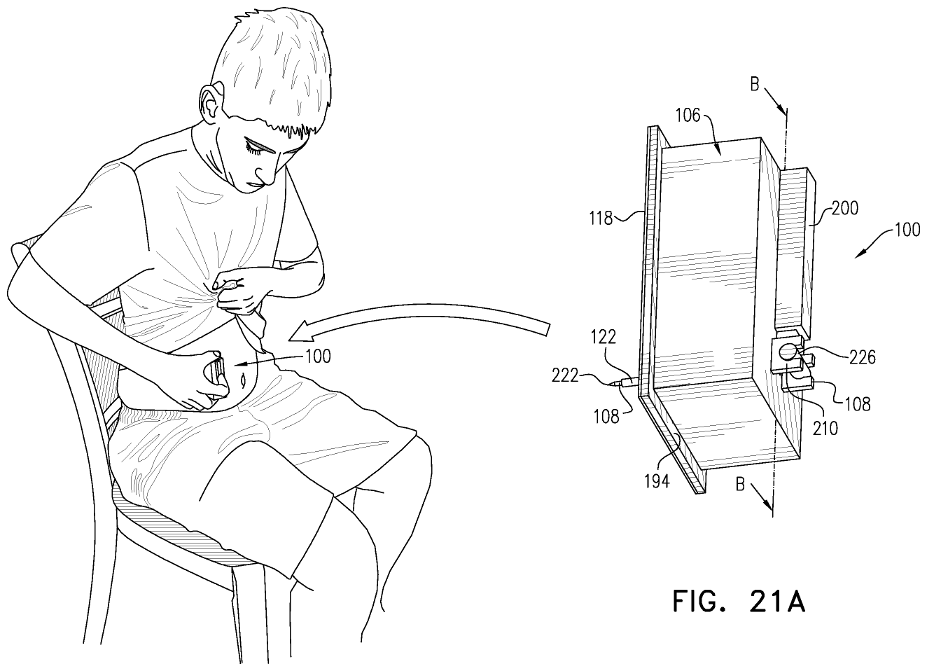

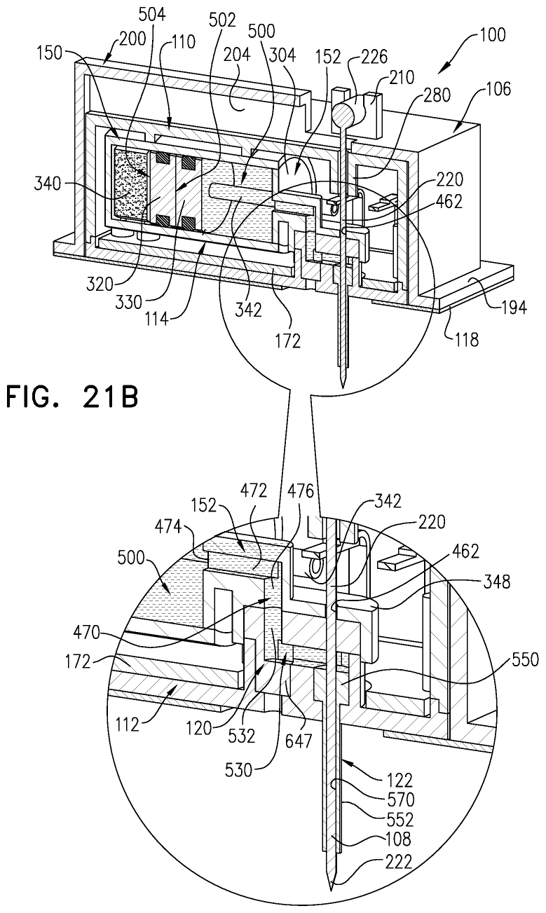

Reference is now made to FIGS. 21A and 21B, which are respective simplified pictorial illustration and sectional view of the transfusion pump 100 of FIG. 1 shown in a fourth operative orientation, section being taken along lines B-B in FIG. 21A.

The transfusion pump 100 is shown in FIGS. 21A & 21B in an injection site attachment operative orientation.

It is seen that the transfusion pump 100 is now turned upside down in comparison with the orientation illustrated and described with reference to FIGS. 20A-20D.

In this injection site attachment operative orientation, it is seen that filling port 102 is now removed from the transfusion pump 100, thereby exposing the penetrating element 108, surrounded by cylindrical portion 552 of cannula 122, while the sharp end 222 of the penetrating element 108 protrudes downwardly from the cannula 122.

In this orientation, the adhesive layer 118 is exposed and the filled transfusion pump 100 along with the inserter 106 are attached to a skin of a patient, while manually inserting the cannula 122 into the injection site by means of the penetrating element 108.

It is appreciated that all remaining spatial relationships between the various components of the transfusion pump 100 as described with reference to FIGS. 20A-20D generally remain unchanged in this fourth operative orientation.

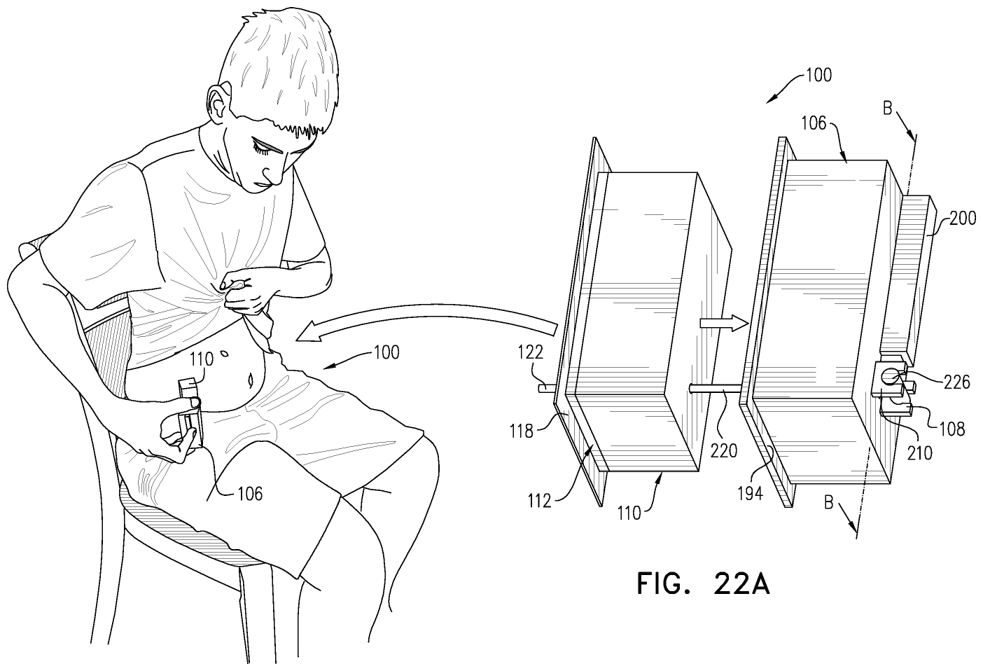

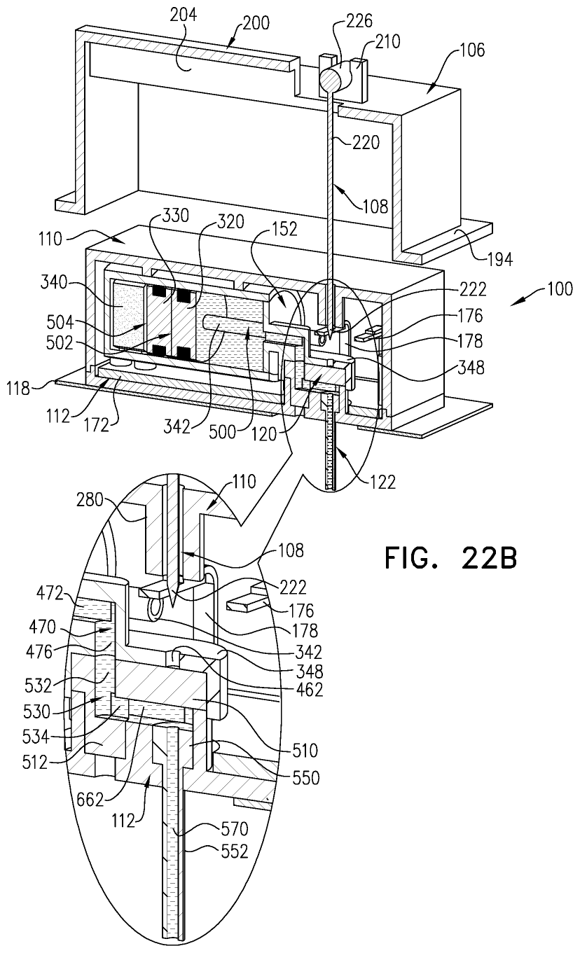

Reference is now made to FIGS. 22A and 22B, which are respective simplified pictorial illustration and sectional view of the transfusion pump 100 of FIG. 1 shown in a fifth operative orientation, section being taken along lines B-B in FIG. 22A.

The transfusion pump 100 is shown in FIGS. 22A & 22B in an inserter removal operative orientation.

In this inserter removal operative orientation, it is seen that the inserter 106 is now partially removed from the transfusion pump 100, such that the top housing portion 110 is not contained within the inserter 106 anymore and the penetrating element 108 is now removed from the cannula 122 and from the flow path septum 120 through aperture 462 and is shown in FIG. 22B as being removed from the protrusion 280 of the top housing portion 110.

In this orientation, fluid flow passage is established between the medication volume 500 and the cannula 122. The base portion 510 of the flow path septum 120 is self-sealed when the penetrating element 108 is removed therefrom and thus medication volume 500 is sealed from the atmosphere.

It is appreciated that all remaining spatial relationships between the various components of the transfusion pump 100 as described with reference to FIGS. 21A & 21B generally remain unchanged in this fifth operative orientation.

Reference is now made to FIGS. 23A and 23B, which are respective simplified pictorial illustration and sectional view of the transfusion pump 100 of FIG. 1 shown in a sixth operative orientation, section being taken along lines B-B in FIG. 23A.

The transfusion pump 100 is shown in FIGS. 23A & 23B in a penetrating element protection operative orientation.

In this penetrating element protection operative orientation, it is seen that the inserter 106 is now fully removed from the transfusion pump 100.

It is a particular feature of an embodiment of the present invention that once the penetrating element 108 is removed from the top housing portion 110 of the transfusion pump 100 it is pivotably biased to its retracted position, in which the penetrating element 108 is disposed within groove 204 of the inserter 106 and is protected therein, preventing accidental pricking of the user.

It is noted that the penetrating element 108 may be pivotably biased to its retracted position by any suitable biasing mechanism, such as torsion spring or leaf spring for example, biasing the penetrating element 108 to be enclosed within the groove 204.

It is appreciated that all remaining spatial relationships between the various components of the transfusion pump 100 as described with reference to FIGS. 22A & 22B generally remain unchanged in this sixth operative orientation.

Reference is now made to FIGS. 24A-24C, which are respective simplified pictorial illustration and two sectional views of the transfusion pump 100 of FIG. 1 shown in a seventh operative orientation, sections being taken along lines B-B and C-C in FIG. 24A. The transfusion pump 100 is shown in FIGS. 24A-24C in an end of injection operative orientation.

In this end of injection operative orientation, it is seen that once the predetermined amount of time has passed, the CPU 600 initiated the injection of medication 704 by activating contacts 173 and 174, which provide current to the pressure generating element 340 and thereby initiate a chemical reaction therein, which in turn produces gas discharge from the pressure generating element 340. Once the pressure generating element 340 is actuated, pressure starts building up within pressure chamber 504 and the volume of the pressure chamber 504, formed between the pressure generating element 340 and the first piston assembly 320 thus increases, thereby axially displaces the first piston assembly 320 forwardly toward the second piston assembly 330.

It is a particular feature of an embodiment of the present invention that at least one of the first piston assembly 320 and the second piston assembly 330 is configured to be driven in a medication pumping displacement direction by the fluid pressure generated by the pressure generating element 340 upon receipt of a suitable signal from the circuit and switch assembly 116.

It is noted that if gas volume 502 was not empty in the previous operative orientation, then the first piston assembly 320 is first displaced forwardly up to engagement of the first piston assembly 320 with the second piston assembly 330 and then the pressure that is built within pressure chamber 504 axially displaces the second piston assembly 330 through the first piston assembly 320 and thus expelling the medication 704 from the medication volume 500 through the fluid flow passage defined by fluid pathway 470 of the reservoir lid 152 and fluid flow path 530 of the flow path septum 120 and further via bore 570 of the cannula 122 into the injection site. It is appreciated that both piston assemblies 320 and 330 are displaced at the same longitudinal direction during medication delivery.

It is seen in FIGS. 24B & 24C that in this end of injection operative orientation, the entire amount of medication 704 contained within medication volume 500 is injected into the body of the patient, such that the second piston assembly 330 abuts the flange 304 of the reservoir lid 152 and the actuating element 342 extends forwardly from flange 304 to its maximal longitudinal extent, such that the actuating element 342 now engages both contacts 614 and 616, thereby closing an electrical circuit therebetween and thus serving as an end switch indicating that the injection is completed.

Once the actuating element 342 is displaced forwardly along with second piston assembly 330, it now extends under the switches 176 and 178 and does not physically interfere therewith.

It is noted that once the actuating element 342 engages contact 616, preferably a visual indication is provided to the user indicating end of injection, by means of LED 606, forming part of the circuit and switch assembly 116. Additionally, an audible indication may be provided to the user as well by means of buzzer 604, forming part of the circuit and switch assembly 116.

It is also noted that in case that following a predetermined amount of time, the actuating element 342 did not engage contact 616 for any reason, then an error alert is presented to the user, indicating that injection did not take place.

It is appreciated that all remaining spatial relationships between the various components of the transfusion pump 100 as described with reference to FIGS. 23A & 23B generally remain unchanged in this seventh operative orientation.

It will be appreciated by persons skilled in the art that the present invention is not limited by what has been particularly shown and described hereinabove, rather the scope of the present invention includes both combinations and subcombinations of various features described hereinabove as well as variations and modifications thereof which are not in the prior art.

1. A pump assembly for pumping a medication through a cannula, said pump assembly comprising:
a housing adapted to be attached to a skin of a patient and containing a reservoir assembly having a variable volume liquid chamber configured to contain said medication upon filling of said reservoir assembly, a variable volume gas chamber configured to contain air before filling of said reservoir assembly and a pressure chamber configured to contain a pressure generating element, said liquid chamber, gas chamber and pressure chamber are fluidly sealed with respect to each other;
said liquid chamber is selectably fluidly couplable with said cannula and said gas chamber is fluidly couplable with the atmosphere.

2. The pump assembly according to claim 1 and wherein a first piston assembly and a second piston assembly being slidably sealingly disposed within said reservoir assembly and wherein said liquid chamber is defined between said second piston assembly and a closed forward end of said reservoir assembly and said gas chamber is defined between said first piston assembly and said second piston assembly.

3. The pump assembly according to claim 2 and also comprising a circuit and switch assembly operative for electronically controlling said pressure generating element.

4-8. (canceled)

9. The pump assembly according to claim 1, also comprising an inserter removably coupled with said pump assembly and having a penetrating element, which is pivotably coupled to said inserter and is configured to extend within said cannula for insertion of said cannula into an injection site;
said penetrating element is pivotable between a retracted position within said inserter and an extended position within said cannula, the penetrating element being biased to said retracted position following removal thereof from said cannula.

10. (canceled)

11. The pump assembly according to claim 1, and wherein a first fluid path is operative for passage of said medicament between said liquid chamber and said cannula and a second fluid path is operative for passage or air between said gas chamber and the atmosphere, and wherein said first fluid path and said second fluid path are fluidly sealed with respect to each other.

12. The pump assembly according to claim 11, and wherein said first fluid path, said second fluid path, said liquid chamber and said gas chamber are accessible for sterilization by a sterilizing agent.

13-26. (canceled)

27. A pump assembly useful for pumping a medication through a cannula,
said pump assembly comprising:
a housing configured for enclosing a reservoir assembly therewithin, said reservoir assembly having a closed forward end, disposed adjacent said cannula;
an electronic assembly, operatively engageable with said reservoir assembly;
said reservoir assembly comprises a first piston assembly, a second piston assembly and a pressure generating element enclosed therewithin, a liquid chamber is configured to be formed between said second piston assembly and said closed forward end; a gas chamber is configured to be formed between said second piston assembly and said first piston assembly and a pressure chamber is configured to be formed between said pressure generating element and said first piston assembly, wherein at least one of said first piston assembly and said second piston assembly is configured to be driven in a medication pumping displacement direction by fluid pressure generated by said pressure generating element upon receipt of a suitable signal from said electronic assembly.

28. The pump assembly according to claim 27, and wherein said at least one of said first piston assembly and said second piston assembly has an actuating element that extends forwardly through said closed forward end and being operative for electrically coupling said pressure generating element to said electronic assembly upon driving of said at least one of said first piston assembly and said second piston assembly in a medication filling displacement direction.

29. The pump assembly according to claim 28, and wherein said housing is adapted to be attached to a skin of a patient and said liquid chamber has a variable volume configured to contain said medication upon filling of said reservoir assembly, said gas chamber has a variable volume configured to contain air before filling of said reservoir assembly and said pressure chamber has a variable volume configured to contain said pressure generating element, said liquid chamber, gas chamber and pressure chamber are fluidly sealed with respect to each other;
said liquid chamber is selectably fluidly couplable with said cannula and said gas chamber is fluidly couplable with the atmosphere.

30. The pump assembly according to claim 27 and wherein said first piston assembly and said second piston assembly being slidably sealingly disposed within said reservoir assembly.

31-32. (canceled)

33. The pump assembly according to claim 27 and wherein said first and second piston assemblies both move in a same axial direction during pumping of said medication through said cannula.

34. (canceled)

35. The pump assembly according to claim 27, also comprising an inserter removably coupled with said pump assembly and having a penetrating element, which is pivotably coupled to said inserter and is configured to extend within said cannula for insertion of said cannula into an injection site;
said penetrating element is pivotable between a retracted position within said inserter and an extended position within said cannula, the penetrating element being biased to said retracted position following removal thereof from said cannula.

36. (canceled)

37. The pump assembly according to claim 27, and wherein a first fluid path is operative for passage of said medicament between said liquid chamber and said cannula and a second fluid path is operative for passage or air between said gas chamber and the atmosphere, and wherein said first fluid path and said second fluid path are fluidly sealed with respect to each other.

38-52. (canceled)

53. A pump assembly for pumping a medication through a cannula, said pump assembly comprising:
a housing adapted to be attached to a skin of a patient and containing a reservoir assembly having a variable volume liquid chamber configured to contain said medication upon filling of said reservoir assembly, a variable volume gas chamber configured to contain air before filling of said reservoir assembly and a pressure chamber configured to contain a pressure generating element, said liquid chamber, gas chamber and pressure chamber are fluidly sealed with respect to each other;
the volume of said liquid chamber is mutually variable with the volume of said gas chamber during filling of said liquid chamber with said medication, such that the volume increase of said liquid chamber corresponds to volume decrease of said gas chamber;
the volume of said liquid chamber is mutually variable with the volume of said pressure chamber during pumping of said medication out of said liquid chamber through said cannula, such that the volume increase of the pressure chamber corresponds to volume decrease of the liquid chamber.

54. The pump assembly according to claim 53 and wherein a first piston assembly is provided between said pressure chamber and said gas chamber; a second piston assembly is provided between said gas chamber and said liquid chamber and said first and second piston assemblies are configured to be slidably sealingly displaceable within said reservoir assembly.

55. The pump assembly according to claim 54, also comprising an electronic assembly, operatively engageable with said reservoir assembly, said reservoir assembly having a closed forward end, disposed adjacent said cannula; said reservoir assembly comprises said first piston assembly, said second piston assembly and a pressure generating element enclosed therewithin, said liquid chamber is configured to be formed between said second piston assembly and said closed forward end; said gas chamber is configured to be formed between said second piston assembly and said first piston assembly and said pressure chamber is configured to be formed between said pressure generating element and said first piston assembly, wherein at least one of said first piston assembly and said second piston assembly is configured to be driven in a medication pumping displacement direction by fluid pressure generated by said pressure generating element upon receipt of a suitable signal from said electronic assembly.

56. (canceled)

57. The pump assembly according to claim 53 and wherein said liquid chamber is selectably fluidly couplable with said cannula and said gas chamber is fluidly couplable with the atmosphere.

58-59. (canceled)

60. The pump assembly according to claim 54 and wherein said first and second piston assemblies both move in a same axial direction during pumping of said medication through said cannula.

61-62. (canceled)

63. The pump assembly according to claim 53, and wherein a first fluid path is operative for passage of said medicament between said liquid chamber and said cannula and a second fluid path is operative for passage or air between said gas chamber and the atmosphere, and wherein said first fluid path and said second fluid path are fluidly sealed with respect to each other.

64-65. (canceled)

66. The pump assembly according to claim 53, also comprising an inserter removably coupled with said pump assembly and having a penetrating element, which is pivotably coupled to said inserter and is configured to extend within said cannula for insertion of said cannula into an injection site;
said penetrating element is pivotable between a retracted position within said inserter and an extended position within said cannula, the penetrating element being biased to said retracted position following removal thereof from said cannula.
